SEQUENCE LISTING (15 sequences)

SEQ ID NO: 1 - protein, 364 aa, synthetic construct
  REGION 1..364: note = Synthetic
QTRRVVLKSAAAAGTLLGGLAGCASVAGSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRAS

SEQ ID NO: 2 - protein, 612 aa, synthetic construct
  REGION 1..612: note = Synthetic
  VARIANT 1..2: note = optionally absent
  VARIANT 180..181: note = optionally absent
  VARIANT 230..248: note = optionally absent
MALDKALVLRTCANNMADHCGLIWPASGTVESRYWQSTRRHENGLVGLLWGAGTSAFLSVHADARWIVCEVAVADIISLEEPGMVKFPRAEVVHVGDRISASHFISARQADPASTSTSTSTSTLTPMPTAIPTPMPAVASVTLPVAEQARHEVFDVASVSAAAAPVNTLPVTTPQNLQTRSRLWDGKRYRQLVARTGENGVEADIPYYVNEDDDIVDKPDEDDDWIEVKSSNNNNNNNNNNLGASGSGQTRRVVLKSAAAAGTLLGGLAGCASVAGSIGTGDRINTVRGPITISEAGFTLTHEHICGSSAGFLRAWPEFFGSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQRDGEQQAAIFESEGLSPSRVCIGHSDDTDDLSYLTALAARGYLIGLDHIPHSAIGLEDNASASALLGIRSWQTRALLIKALIDQGYMKQILVSNDWLFGFSSYVTNIMDVMDRVNPDGMAFIPLRVIPFLREKGVPQETLAGITVTNPARFLSPTLRAS

SEQ ID NO: 3 - protein, 384 aa, synthetic construct
  REGION 1..384: note = Synthetic
  VARIANT 1: note = optionally absent
MQTRRDALKSAAAITLLGGLAGCASMARPIGTGDLINTVRGPIPVSEAGFTLTHEHICGSSAGFLRAWPEFFGSRKALAEKAVRGLRHARSAGVQTIVDVSTFDIGRDVRLLAEVSRAADVHIVAATGLWFDPPLSMRMRSVEELTQFFLREIQHGIEDTGIRAGIIKVATTGKATPFQELVLKAAARASLATGVPVTTHTSASQRDGEQQAAIFESEGLSPSRVCIGHSDDTDDLSYLTGLAARGYLVGLDRMPYSAIGLEGNASALALFGTRSWQTRALLIKALIDRGYKDRILVSHDWLFGFSSYVTNIMDVMDRINPDGMAFVPLRVIPFLREKGVPPETLAGVTVANPARFLSPTVRAVVTRSETSRPAAPIPRQDTER

SEQ ID NO: 4 - protein, 330 aa, synthetic construct
  REGION 1..330: note = Synthetic
  VARIANT 1: note = optionally absent
MPLKNRLLARLSCVAAVVAATAAVAPLTLVSTAHAAAPQVRTSAPGYYRMLLGDFEITALSDGTVALPVDKRLNQPAPKTQSALAKSFQKAPLETSVTGYLVNTGSKLVLVDTGAAGLFGPTLGRLAANLKAAGYQPEQVDEIYITHMHPDHVGGLMVGEQLAFPNAVVRADQKEADFWLSQTNLDKAPDDESKGFFKGAMASLNPYVKAGKFKPFSGNTDLVPGIKALASHGHTPGHTTYVVESQGQKLALLGDLILVAAVQFDDPSVTTQLDSDSKSVAVERKKAFADAAKGGYLIAASHLSFPGIGHIRAEGKGYRFVPVNYSVVNP

SEQ ID NO: 5 - protein, 314 aa, synthetic construct
  REGION 1..314: note = Synthetic
  VARIANT 1: note = optionally absent
MRIPLVGKDSIESKDIGFTLIHEHLRVFSEAVRQQWPHLYNEDEEFRNAVNEVKRAMQFGVKTIVDPTVMGLGRDIRFMEKVVKATGINLVAGTGIYIYIDLPFYFLNRSIDEIADLFIHDIKEGIQGTLNKAGFVKIAADEPGITKDVEKVIRAAAIANKETKVPIITHSNAHNNTGLEQQRILTEEGVDPGKILIGHLGDTDNIDYIKKIADKGSFIGLDRYGLDLFLPVDKRNETTLRLIKDGYSDKIMISHDYCCTIDWGTAKPEYKPKLAPRWSITLIFEDTIPFLKRNGVNEEVIATIFKENPKKFFS

SEQ ID NO: 6 - protein, 229 aa, synthetic construct
  REGION 1..229: note = Synthetic
  VARIANT 1..2: note = optionally absent
  VARIANT 180..181: note = optionally absent
MALDKALVLRTCANNMADHCGLIWPASGTVESRYWQSTRRHENGLVGLLWGAGTSAFLSVHADARWIVCEVAVADIISLEEPGMVKFPRAEVVHVGDRISASHFISARQADPASTSTSTSTSTLTPMPTAIPTPMPAVASVTLPVAEQARHEVFDVASVSAAAAPVNTLPVTTPQNLQTRSRLWDGKRYRQLVARTGENGVEADIPYYVNEDDDIVDKPDEDDDWIEVK

SEQ ID NO: 7 - protein, 146 aa, synthetic construct
  REGION 1..146: note = Synthetic
KATKLVLGAVILGSTLLAGCSSNAKIDQGINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRPW

SEQ ID NO: 8 - protein, 563 aa, synthetic construct
  REGION 1..563: note = Synthetic
  VARIANT 1: note = optionally absent
MPIKYKPEIQHSDFKDLTNLIHFQSMEGKIWLGEQRMLLLQFSAMASFRREMVNTLGIERAKGLFLRHGYQSGLKDAELARKLRPNASEVGMFLAGPQMHSLKGLVKVRPTELDIDKEYGRFYAEMEWIDSFEVEICQTDLGQMQDPVCWTLLGYACAYSSAFMGREIIFKEVSCRGCGGDKCRVIGKPAEEWDDVASFKQYFKNDPIIEELYELQSQLLSLRTNLDKQEGQYYGIGQTPAYQTVRNMMDKAAQGKVSVLLLGETGVGKEVIARSVHLRSKRAAEPFVAVNCAAIPPDLIESELFGVEKGAFTGATQSRMGRFERADKGTIFLDEVIELSPRAQASLLRVLQEGELERVGDNRTRKIDVRVIAATHEDLAEAVKAGRFRADLYYRLNVFPVAIPALRERREDIPLLVEHFLQRFHQEYGKRTLGLSDKALEACLHYSWPGNIRELENVIERGIILTDPNESISVQALFLRAPEEPQTASERVSLDGVLIQPGNGQGSWISQLLSSGLSLDEIEESLMREAMQQANQNVSGAARLLGLSRPALAYRLKKIGIEG

SEQ ID NO: 9 - protein, 187 aa, synthetic construct
  REGION 1..187: note = Synthetic
  VARIANT 1: note = optionally absent
MNWRALFKPSAKYSILALLVVGIVIGVVGYFATQQTLHATSTDAFCMSCHSNHSLKNEVLASAHGGGKAGVTVQCQDCHLPHGPVDYLIKKIIVSKDLYGFLTIDGFNTQAWLDENRKEQADKALAYFRGNDSANCQHCHTRIYENQPETMKPMAVRMHTNNFKKDPETRKTCVDCHKGVAHPYPKG

SEQ ID NO: 10 - protein, 333 aa, synthetic construct [sequence omitted]

SEQ ID NO: 11 - protein, 697 aa, synthetic construct
  REGION 1..697: note = Synthetic
  VARIANT 1: note = optionally absent
MKFKLNLITLALLANTGLAVAADGYGLANANTEKVKLSAWSCKGCVVETGTSGTVGVGVGYNSEEDIRSANAFGTSNEVAGKFDADLNFKGEKGYRASVDAYQLGMDGGRLDVNAGKQGQYNVNVNYRQIATYDSNSALSPYAGIGGNNLTLPDNWITAGSSNQMPLLMDSLNALELSLKRERTGLGFEYQGESLWSTYVNYMREEKTGLKQASGSFFNQSMMLAEPVDYTTDTIEAGVKLKGDRWFTALSYNGSIFKNEYNQLDFENAFNPTFGAQTQGTMALDPDNQSHTVSLMGQYNDGSNALSGRILTGQMSQDQALVTDNYRYANQLNTDAVDAKVDLLGMNLKVVSKVSNDLRLTGSYDYYDRDNNTQVEEWTQISINNVNGKVAYNTPYDNRTQRFKVAADYRITRDIKLDGGYDFKRDQRDYQDRETTDENTVWARLRVNSFDTWDMWVKGSYGNRDGSQYQASEWTSSETNSLLRKYNLADRDRTQVEARITHSPLESLTIDVGARYALDDYTDTVIGLTESKDTSYDANISYMITADLLATAFYNYQTIESEQAGSSNYSTPTWTGFIEDQVDVVGAGISYNNLLENKLRLGLDYTYSNSDSNTQVRQGITGDYGDYFAKVHNINLYAQYQATEKLALRFDYKIENYKDNDAANDIAVDGIWNVVGFGSNSHDYTAQMLMLSMSYKL

SEQ ID NO: 12 - protein, 671 aa, synthetic construct
  REGION 1..671: note = Synthetic
MMNAQKSKIALLLAASAVTMALTGCGGSDGNNGNDGSDGGEPAGSIQTLNLDITKVSYENGAPMVTVFATNEADMPVIGLANLEIKKALQLIPEGATGPGNSANWQGLGSSKSYVDNKNGSYTFKFDAFDSNKVFNAQLTQRFNVVSAAGKLADGTTVPVAEMVEDFDGQGNAPQYTKNIVSHEVCASCHVEGEKIYHQATEVETCISCHTQEFADGRGKPHVAFSHLIHNVHNANKAWGKDNKIPTVAQNIVQDNCQVCHVESDMLTEAKNWSRIPTMEVCSSCHVDIDFAAGKGHSQQLDNSNCIACHNSDWTAELHTAKTTATKNLINQYGIETTSTINTETKAATISVQVVDANGTAVDLKTILPKVQRLEIITNVGPNNATLGYSGKDSIFAIKNGALDPKATINDAGKLVYTTTKDLKLGQNGADSDTAFSFVGWSMCSSEGKFVDCADPAFDGVDVTKYTGMKADLAFATLSGKAPSTRHVDSVNMTACANCHTAEFEIHKGKQHAGFVMTEQLSHTQDANGKAIVGLDACVTCHTPDGTYSFANRGALELKLHKKHVEDAYGLIGGNCASCHSDFNLESFKKKGALNTAAAADKTGLYSTPITATCTTCHTVGSQYMVHTKETLESFGAVVDGTKDDATSAAQSETCFYCHTPTVADHTKVKM

SEQ ID NO: 13 - protein, 571 aa, synthetic construct
  REGION 1..571: note = Synthetic
ADNLAEFHVQNQECDSCHTPDGELSNDSLTYENTQCVSCHGTLEEVAETTKHEHYNAHASHFPGEVACTSCHSAHEKSMVYCDSCHSFDFNMPYAKKWQRDEPTIAELAKDKSERQAALASAPHDTVDVVVVGSGGAGFSAAISATDSGAKVILIEKEPVIGGNAKLAAGGMNAAWTDQQKAKKITDSPELMFEDTMKGGQNINDPALVKVLSSHSKDSVDWMTAMGADLTDVGMMGGASVNRAHRPTGGAGVGAHVVQVLYDNAVKRNIDLRMNTRGIEVLKDDKGTVKGILVKGMYKGYYWVKADAVILATGGFAKNNERVAKLDPSLKGFISTNQPGAVGDGLDVAENAGGALKDMQYIQAHPTLSVKGGVMVTEAVRGNGAILVNREGKRFVNEITTRDKASAAILAQTGKSAYLIFDDSVRKSLSKIDKYIGLGVAPTADSLVKLGKMEGIDGKALTETVARYNSLVSSGKDTDFERPNLPRALNEGNYYAIEVTPGVHHTMGGVMIDTKAEVMNAKKQVIPGLYGAGEVTGGVHGANRLGGNAISDIITFGRLAGEEAAKYSKKN

SEQ ID NO: 14 - DNA, 199 bp, synthetic construct
  misc_feature 1..199: note = Synthetic
  source 1..199: mol_type = other DNA
taagcatttgctcaagcggccttgggcaattgatcaaatgcttaaaaagtctgcgcaagcgcggcttaatttcgctcgctccgatcattctaaaaattagaaacacattgaaaaacattaccttgaagtctgttttcagaccttggcacagccgttgcttgatgtcctgcgtactagagtcacacaggaaaccactaga

SEQ ID NO: 15 - DNA, 929 bp, synthetic construct
  misc_feature 1..929: note = Synthetic
  source 1..929: mol_type = other DNA
taagcatttgctcaagcggccttgggcaattgatcaaatgcttaaaaagtctgcgcaagcgcggcttaatttcgctcgctccgatcattctaaaaattagaaacacattgaaaaacattaccttgaagtctgttttcagaccttggcacagccgttgcttgatgtcctgcgtactagagtcacacaggaaaccactagaagtcataagtctgggctaagcccactgatgagtcgctgaaatgcgacgaaacttatgacctctacaaataattttgtttaattcgctttgggtttttaaggaggacgcaatgaactggcgtgcactatttaaacccagcgcgaaatattccatcctagcgctactggttgttggtatcgtgattggtgttgtgggctattttgcaactcagcagactttacatgcgacaagtacagatgcgttctgtatgtcttgccatagcaatcattccttgaagaatgaagtgctggcatctgcccacggtggcggcaaagccggggttactgttcagtgtcaagactgtcacttaccccatggccctgttgattatttaattaagaaaatcatcgtatctaaagatttatatggtttcttaactattgatggctttaacactcaagcttggttagacgaaaaccgcaaagagcaagccgacaaagcattggcttacttccgtggtaacgactcagcaaactgtcaacactgccatactcgcatttatgaaaaccagccagaaaccatgaagccaatggctgtgagaatgcacaccaacaacttcaagaaagatcctgaaacgagaaagacctgtgtggattgccacaaaggtgtcgctcacccctatccaaaaggataactcggtaccaaagacgaacaataagacgctgaaaagcgtcttttttcgttttggtcc

(19) United States
(12) Patent Application Publication
(10) Pub. No.: US 2024/0368534 A1
(43) Pub. Date: Nov. 7, 2024

(54) A MICROBIAL ELECTROCHEMICAL TECHNOLOGY TO DETECT AND DEGRADE ORGANOPHOSPHATE PESTICIDES

(71) Applicant: MASSACHUSETTS INSTITUTE OF TECHNOLOGY, Cambridge, MA (US)

(72) Inventors: Ariel FURST, Cambridge, MT (US); Rachel AHLMARK, Lansdale, PA (US); Amruta KARBELKAR, White River Junction, VT (US); Erin REYNOLDS, Westborough, MA (US)

(21) Appl. No.: 18/690,033

(22) PCT Filed: Sep. 14, 2022
(86) PCT No.: PCT/US2022/076432

§ 371 Date: Mar. 7, 2024

Related U.S. Application Data
(60) Provisional application No. 63/244,566, filed on Sep. 15, 2021.

(51) Int. Cl. C12N 1/20; A62D 3/02; C12N 9/16; C12R 1/19

(52) U.S. Cl. CPC C12N 1/20; A62D 3/02; C12N 9/16; C12R 2001/19; C12Y 301/08001

(57) ABSTRACT

Provided herein are recombinant microbial cells displaying on their surface a non-native protein capable of degrading an organophosphate, wherein the recombinant microbial cell has inhibited replication, as well as recombinant microbial cells engineered to be capable of expressing a non-native transcription factor that activates a non-native promoter in response to an organophosphate degradation product, wherein the non-native promoter is operatively linked to a nucleic acid encoding a reporter protein, wherein activity of the reporter protein can be detected, and their use for degrading organophosphates and detecting organophosphate degradation products.

### CROSS REFERENCE

This application claims priority to U.S. Provisional Patent Application Ser. No. 63/244,566 filed Sep. 15, 2021, incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on Aug. 30, 2022 having the file name “21-1511-WO.xml” and is 30 kb in size.

### BACKGROUND

Organophosphate (OP) pesticides cause hundreds of illnesses and deaths annually. Unfortunately, exposures are often detected by monitoring degradation products in blood and urine, with few effective methods for detection and remediation at the point of dispersal. The organophosphate (OP) pesticides parathion and paraoxon cause thousands of illnesses and deaths annually, as they have the same mechanism of action as the nerve gas sarin. Such compounds disrupt the native neurotransmitter acetylcholine and impact the parasympathetic nerve system, which can be deadly. Similar compounds were employed as chemical weapons during World War II in the form of sarin, cyclosarin, and soman. OP chemical warfare agents (CWAs) have continued to be used as recently as 2017.

The main application of OPs is not as CWAs but as insecticides and pesticides. In fact, OPs are the most widely-used pesticides in industrialized countries, causing environmental contamination and posing significant danger following exposure. Annually, there are an estimated three million exposures to OPs, causing 300,000 fatalities. The continued danger of these pesticides necessitates vigilance and affordable, easy-to-use technologies for detection and remediation, especially in low resource settings.

### SUMMARY

In a first aspect, the disclosure provides recombinant microbial cells displaying on its surface a non-native protein capable of degrading an organophosphate, wherein the recombinant microbial cell has inhibited replication. In one embodiment, the recombinant cell is treated to reduce its ability to replicate by chemical treatment including but not limited to treatment with sodium azide or by lyophilization. In another embodiment, the protein comprises a phosphate hydrolase, beta-lactamases-methyl parathion hydrolase, lactonase, or a phosphate triesterase, or an enzymatically active fragment thereof. In one embodiment, the protein may be selected from the group including, but not limited to an organophosphate hydrolase (OPH), OP (organophosphate)-degrading enzyme from Agrobacterium radiobacter (OpdA), phosphotriesterase (PTE), methyl parathion hydrolase (MPH), SsoPOX, or an enzymatically active fragment thereof. In a further embodiment, the protein comprises an amino acid sequence at least 75%, 80%, 85%, 90%, 91%, 92%, 93%, 94%, 95%, 96%, 97%, 98%, 99%, or 100% identical to the amino acid sequence of SEQ ID NO: 1 or 3-5, wherein residues in parentheses are optional and may be present or absent.

In one embodiment, the protein further comprise one or more domain that facilitates microbial cell surface expression of the protein. In another embodiment, the domain comprises INPNC ice-nucleation sequence (see, for example, U.S. Pat. No. 8,759,028, incorporated by reference herein in its entirety) or OmpA signal peptide. In a further embodiment, the domain comprises an amino acid sequence at least 75%, 80%, 85%, 90%, 91%, 92%, 93%, 94%, 95%, 96%, 97%, 98%, 99%, or 100% identical to the amino acid sequence of SEQ ID NO:6 or 7, wherein residues in parentheses are optional and may be present or absent, and when present may be substituted with any other amino acid linker of the same or different length and amino acid composition.

In one embodiment, the protein comprises an amino acid sequence at least 75%, 80%, 85%, 90%, 91%, 92%, 93%, 94%, 95%, 96%, 97%, 98%, 99%, or 100% identical to the amino acid sequence of SEQ ID NO:2, wherein the amino acid residues in parentheses are optional and may be present or absent, and when present may be substituted with any other amino acid linker of the same or different length and amino acid composition. In another embodiment, the protein is encoded by a nucleic acid coding sequence present on a plasmid in the microbial cell, wherein the nucleic acid is operatively linked to control sequence capable of promoting expression of the nucleic acid. In a further embodiment, the microbial cell is a bacteria, including but not limited to E.coli. In one embodiment, the cell is E. coli displaying an enzymatically active portion of parathion hydrolase from Pseudomonas diminuta on its surface, including but not limited to an enzymatically active portion of the amino acid sequence of SEQ ID NO:1 on its surface. In another embodiment, the cell displays an enzymatically active portion of the amino acid sequence of SEQ ID NO:2 on its surface.

In a second aspect, the disclosure provides recombinant microbial cells engineered to be capable of expressing a non-native transcription factor that activates a non-native promoter in response to an organophosphate degradation product; wherein the non-native promoter is operatively linked to a nucleic acid encoding a reporter protein, wherein activity of the reporter protein can be detected. In one embodiment, the non-native transcription factor comprises DmpR, or variants thereof, or wherein the non-native transcription factor comprises any small molecule-responsive transcription factor. In another embodiment, the transcription factor comprises an amino acid sequence at least 75%, 80%, 85%, 90%, 91%, 92%, 93%, 94%, 95%, 96%, 97%, 98%, 99%, or 100% identical to the amino acid sequence of SEQ ID NO: 8, optionally modified with mutations Q10R and K117M, wherein residues in parentheses are optional and may be present or absent.

In one embodiment, the recombinant microbial cell comprises a plasmid that comprises (a) a nucleic acid encoding the non-native transcription factor and (b) the non-native promoter operatively linked to the reporter protein. In one embodiment, a nucleic acid encoding the non-native transcription factor is operatively linked to a constitutively active promoter, wherein the promoter may optionally comprises the nucleic acid sequence of SEQ ID NO: 14. In another embodiment, the reporter protein comprises a fluorescent protein and/or a protein component of an extracellular electron transfer system. In various embodiments, the reporter protein may comprise Cytochrome c-type protein (CymA), Extracellular iron oxide respiratory system outer membrane component MtrA (MtrA), Extracellular iron oxide respiratory system outer membrane component MtrB (MtrB), Extracellular iron oxide respiratory system surface decaheme cytochrome c component MtrC (MtrC), Fumarate reductase flavoprotein subunit (FccA), or a functional fragment thereof. In other embodiments, the reporter protein may comprise an amino acid sequence at least 75%, 80%, 85%, 90%, 91%, 92%, 93%, 94%, 95%, 96%, 97%, 98%, 99%, or 100% identical to the amino acid sequence of any one of SEQ ID NO:9-13, wherein residues in parentheses are optional and may be present or absent.

In one embodiment, the microbial cell is a bacteria; in other embodiments, the microbial cell may be any Shewanella species including but not limited to S. oneidensis, or any species of Geobacter. In another embodiment, the cell is S. oneidensis comprising a nucleic acid encoding non-native transcription factor DmpR comprising the amino acid sequence of SEQ ID NO: 8, or a modified version thereof (such as modified with mutations Q10R and K117M) wherein residues in parentheses are optional and may be present or absent, operatively linked to a constitutive promoter, wherein the promoter may optionally comprises the nucleic acid sequence of SEQ ID NO: 14; wherein the reporter protein comprises an amino acid sequence comprising the amino acid sequence of SEQ ID NO: 9 (CymA) or a functional fragment thereof, wherein residues in parentheses are optional and may be present or absent.

The disclosure also provides kits comprising a first recombinant microbial cell comprising the recombinant microbial cell of any embodiment of the first aspect of the disclosure, and a second recombinant microbial cell comprising the recombinant microbial cell of any embodiment of the second aspect of the disclosure.

The disclosure also provides methods for degrading organophosphates, comprising contacting a source suspected of containing organophosphates with a first recombinant microbial cell of any embodiment of the first aspect of the disclosure, thereby degrading organophosphates present in the source.

The disclosure further provides methods for degradation of organophosphates and detection of organophosphate degradation products, comprising
(a) contacting a source suspected of containing organophosphates with (i) a first recombinant microbial cell of any embodiment of the first aspect of the disclosure, thereby degrading organophosphates present in the source to generate organophosphate degradation products; and (ii) a second recombinant microbial cell of any embodiment of the second aspect of the disclosure;
(b) detecting reporter protein signal, wherein the reporter protein signal provides a measure of organophosphate degradation products in the source.

### DESCRIPTION OF THE FIGURES

FIG. 1. Degradation of organophosphates (OPs) by engineered E. coli. a) Schematic representation of paraoxon degradation to p-nitrophenol (p-NP) by organophosphate hydrolase (OPH)-expressing E. coli and structures of OPs analyzed in this study, namely, paraoxon, paraoxon-methyl, parathion, and malaoxon. b) Production of p-NP over time by OPH-expressing E. coli at different paraoxon concentrations, ranging from 5 to 200 μM c) Effect of paraoxon concentration on the initial rate of p-NP production, fitted with Michaelis-Menten equation. d) Effect of OPH-expressing E. coli cell concentration (expressed in terms of OD₆₀₀) on the initial rate of p-NP production, fitted with a linear regression line, R²=0.99 (black). Error bars represent SD for n=3 biological replicates.

FIG. 2. Detection of organophosphate (OP) degradation product, p-nitrophenol (p-NP) by engineered S. oneidensis. a) Scheme depicting the genetic circuit engineered in S. oneidensis in which p-NP binds to the transcription factor DmpR, inducing the expression of Green Fluorescent Protein (GFP). b) GFP fluorescence in S. oneidensis cultures with and without the inducer, p-NP (left). Scheme depicting an electrochemical bioreactor set-up containing carbon cloth working electrode, Pt counter electrode and AgCl/Ag reference electrode (right). c) GFP fluorescence normalized to OD₆₀₀ produced by engineered S. oneidensis in response to different concentrations of p-NP and paraoxon, and by wild-type S. oneidensis cells with different p-NP concentrations after 24 h induction. d) Scheme depicting genetic circuit engineered in S. oneidensis in which p-NP binds to DmpR, inducing the expression of CymA, a critical extracellular electron transfer protein in S. oneidensis. c) Representative chronoamperometry plots indicating the change in current density over time in bioreactors containing engineered S. oneidensis cells with 20 μM p-NP (dark green), cells with 20 μM paraoxon, cells without any inducer and 20 μM p-NP without cells. f) Total charge produced by the cells in 24 h during chronoamperometry experiments under varying conditions. Error bars represent SD for n=3 biological replicates.

FIG. 3. GFP fluorescence response of engineered co-cultures to OPs. a) Scheme depicting the degradation of paraoxon by OPH-expressing E. coli to produce p-NP, which is detected by engineered S. oneidensis, causing GFP expression in cells. b) GFP fluorescence normalized to OD₆₀₀ produced over time by engineered co-cultures in the presence of different initial concentrations of paraoxon, ranging from 0 to 150 μM and co-culture with wild-type E. coli containing 50 μM paraoxon. Error bars represent SD for n=3 replicates.

FIG. 4. Electrochemical response of engineered co-cultures to organophosphates. a) Scheme depicting the degradation of paraoxon by OPH-expressing E. coli to p-NP, which further induces expression of CymA in engineered S. oneidensis, allowing EET from the cells to an external electrode. b) Representative chronoamperometry plots indicating the change in current density over time in bioreactors containing engineered co-cultures with 20 μM paraoxon, S. oneidensis with 20 μM paraoxon, co-culture only and 20 μM paraoxon without cells. c) Total charge produced by the cells in 24 h during chronoamperometry experiments under different conditions. d) Total charge produced by the engineered co-cultures in 24 h during chronoamperometry experiments in the presence of different organophosphates. c) Total charge produced by the co-cultures over time during chronoamperometry experiments with different initial paraoxon concentrations ranging from 0 to 20 μM. Error bars represent SD for n=3 replicates.

FIG. 5. Production of p-NP over time by OPH-expressing E. coli at different cell densities represented as OD₆₀₀. Error bars represent SD for n=3 replicates.

FIG. 6. Production of p-NP over time by OPH-expressing E. coli at different organophosphate concentrations for a) parathion and b) paraoxon-methyl. Effect of organophosphate concentration on the initial rate of p-NP production, fitted with Michaelis-Menten equation for c) parathion and d) paraoxon-methyl. Error bars represent SD for n=3 replicates

FIG. 7. GFP fluorescence normalized by OD₆₀₀ produced by p-NP responsive GFP-expressing E. coli strain in response to different concentrations of p-NP and paraoxon, and by wild-type E. coli cells with different p-NP concentrations after 24 h induction. Error bars represent SD for n=3 replicates.

FIG. 8. GFP fluorescence normalized by OD₆₀₀ produced by engineered S. oneidensis in response to different concentrations of p-NP, dopamine, hydroquinone and 4-methylcatechol after 24 h induction. Error bars represent SD for n=3 replicates

FIG. 9. Raw data from representative chronoamperometry plots indicating the change in current density over time in bioreactors containing engineered S. oneidensis with 20 μM p-NP, cells with 20 μM paraoxon, cells without any inducer and 20 μM p-NP without cells.

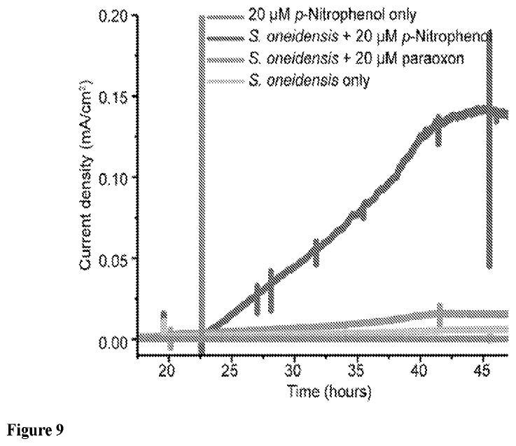

FIG. 10. Normalized GFP fluorescence produced over time by engineered cell mixtures (OPH-expressing E. coli with p-NP inducible E. coli) in the presence of different initial concentrations of paraoxon, ranging from 0 to 150 μM and co-culture with wild-type E. coli containing 50 μM paraoxon. Error bars represent SD for n=3 replicates.

FIG. 11. Normalized GFP fluorescence produced over time by engineered co-cultures (OPH-expressing E. coli with p-NP inducible S. oneidensis) in the presence of different initial concentrations of OPs, ranging from 0 to 150 μM and co-culture with wild-type E. coli containing 50 μM organophosphate for a) paraoxon-methyl b) parathion c) malaoxon. Error bars represent SD for n=3 replicates.

FIG. 12. Raw data from representative chronoamperometry plots indicating the change in current density over time in bioreactors containing engineered co-cultures with 20 μM paraoxon, S. oneidensis with 20 μM paraoxon, co-culture only and 20 μM paraoxon without cells.

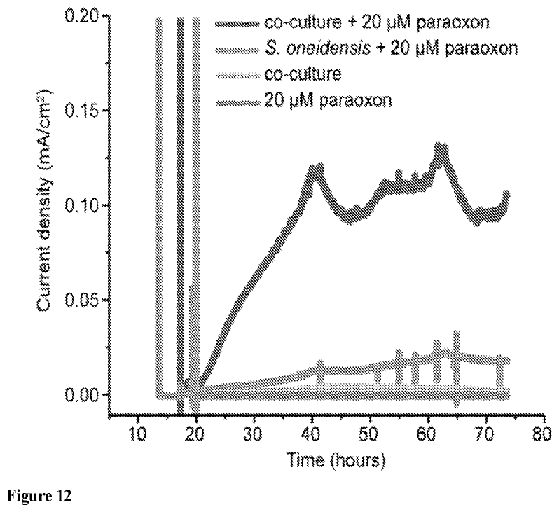

FIG. 13. Representative chronoamperometry plots indicating the change in current density over time in bioreactors containing engineered co-cultures with 20 μM paraoxon-methyl, 20 μM parathion, 20 μM malaoxon.

FIG. 14. a) Representative chronoamperometry plots indicating the change in current density over time in bioreactors containing engineered co-cultures with different concentrations of paraoxon, ranging from 0 to 20 μM. b) Effect of paraoxon concentration over the total charge accumulated by the co-culture in 24 h, fitted with a linear regression line from 5-20 μM.

### DETAILED DESCRIPTION

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the invention can be used in combination, unless the context clearly dictates otherwise.

In all embodiments of polypeptides disclosed herein, any N-terminal methionine residues are optional (i.e.: the N-terminal methionine residue may be present or may be absent, and may be included or excluded when determining percent amino acid sequence identity compared to another polypeptide).

In all embodiments of polypeptides disclosed herein, 1, 2, 3, 4, or 5 amino acids may be deleted from the N-terminus and/or the C-terminus so long as function is maintained, and not be considered when determining percent identity.

In a first aspect, the disclosure provides recombinant microbial cells displaying on their surface a non-native protein capable of degrading an organophosphate, wherein the recombinant microbial cell has inhibited replication.

By “non-native protein” is meant that the protein is not normally expressed in the microbial cell. By “microbial/microbe” is meant bacteria, archaea, protozoa, algae. As used herein an organophosphate is any organic compound whose molecule contains one or more phosphate ester or phosphorothiolate ester groups. By “inhibited replication” is meant that the cell is treated to reduce its ability to replicate relative to the untreated microbial cell.

The recombinant microbial cell of this aspect of the disclosure provides a scaffold for displaying an active enzyme even when the microbial cells are rendered incapable of replication, enabling the cells to serve only as an inert scaffold and permitting their use, for example, in a co-culture organophosphate (“OP”) degradation product system. The microbial cells of this aspect of the disclosure circumvent a core challenge in such co-culture design: maintaining the viability of two microbial strains simultaneously. Using such an engineered microbial system, the inventors have demonstrated detection of OP degradation products at parts per million levels, outperforming reported absorbance and fluorescence sensors.

In one embodiment, the recombinant cell is treated to reduce its ability to replicate by chemical treatment including but not limited to treatment with sodium azide, or by lyophilization. In one embodiment, the recombinant cell is lyophilized.

The disclosure also provides recombinant microbial cell displaying on its surface a non-native protein capable of degrading an organophosphate, wherein the recombinant microbial cell is lyophilized.

The microbial cells of the disclosure may comprise any non-native protein capable of degrading an organophosphate as deemed appropriate for an intended purpose. In various non-limiting embodiments, the protein is capable of degrading an organophosphate selected from the group consisting of parathion, paraoxon, paraoxon-methyl, sarin, cyclosarin, malathion, diazinon, fenthion, dichlorvos, chlorpyrifos, tetrachlorvinfos, oxydemeton methyl, ethion, phosmet, soman, tabun, VX and soman,

Examples of organophosphates include the following:
Insecticides—Malathion, parathion, diazinon, fenthion, dichlorvos, chlorpyrifos, ethion.
Nerve gases—Soman, sarin, tabun, VX.

In various non-limiting embodiments, the protein comprises a phosphate hydrolase, beta-lactamases-methyl parathion hydrolase, lactonase, or a phosphate triesterase, or an enzymatically active fragment thereof. In these embodiments, the protein may comprise any phosphate hydrolase, beta-lactamases-methyl parathion hydrolase, lactonase, or a phosphate triesterase as deemed appropriate for an intended use. In various non-limiting embodiments, the protein may be selected from the group including, but not limited to an organophosphate hydrolase (OPH), OP (organophosphate)-degrading enzyme from Agrobacterium radiobacter (OpdA), phosphotriesterase (PTE), methyl parathion hydrolase (MPH), SsoPox, or an enzymatically active fragment thereof. In these embodiments, the protein may comprise any such protein as deemed suitable for an intended purpose. In one embodiment, the protein a phosphotriesterase obtained from Pseudomonas diminuta, including but not limited to parathion hydrolase, or an enzymatically active fragment thereof. In various non-limiting embodiments, the protein comprises an amino acid sequence at least 75%, 80%, 85%, 90%, 91%, 92%, 93%, 94%, 95%, 96%, 97%, 98%, 99%, or 100% identical to the amino acid sequence selected from SEQ ID NO:1, and 3-5, wherein residues in parentheses are optional and may be present or absent.

Parathion hydrolase from Pseudomonas diminuta (Uniprot ID: P0A434):
(SEQ ID NO: 1)
QTRRVVLKSAAAAGTLLGGLAGCASVAGSIGTGDRINTVRGPITISEAGFTLTHEHICGSSAGFLRAWPEFFGSR
KALAEKAVRGLRRARAAGVRTIVDVSTFDIGRDVSLLAEVSRAADVHIVAATGLWEDPPLSMRLRSVEELTQFFL
REIQYGIEDTGIRAGIIKVATTGKATPFQELVLKAAARASLATGVPVTTHTAASQRDGEQQAAIFESEGLSPSRV
CIGHSDDTDDLSYLTALAARGYLIGLDHIPHSAIGLEDNASASALLGIRSWQTRALLIKALIDQGYMKQILVSND
WLFGFSSYVTNIMDVMDRVNPDGMAFIPLRVIPFLREKGVPQETLAGITVTNPARFLSPTLRAS
OpdA embodiment: Rhizobium radiobacter (Agrobacterium tumefaciens)
(Agrobacterium radiobacter)
>tr|Q93LD7|Q93LD7_RHIRD Phosphotriesterase OS = Rhizobium radiobacter OX = 358
GN = opdA PE = 1 SV = 1
(SEQ ID NO: 3)
(M)QTRRDALKSAAAITLLGGLAGCASMARPIGTGDLINTVRGPIPVSEAGFTLTHEHICGS
SAGFLRAWPEFFGSRKALAEKAVRGLRHARSAGVQTIVDVSTFDIGRDVRLLAEVSRAAD
VHIVAATGLWFDPPLSMRMRSVEELTQFFLREIQHGIEDTGIRAGIIKVATTGKATPFQE
LVLKAAARASLATGVPVTTHTSASQRDGEQQAAIFESEGLSPSRVCIGHSDDTDDLSYLT
GLAARGYLVGLDRMPYSAIGLEGNASALALFGTRSWQTRALLIKALIDRGYKDRILVSHD
WLFGFSSYVTNIMDVMDRINPDGMAFVPLRVIPFLREKGVPPETLAGVTVANPARFLSPT
VRAVVTRSETSRPAAPIPRQDTER
Methyl parathion hydrolase embodiment:
Pseudomonas sp. (strain WBC-3):
>tr|Q841S6|Q841S6_PSEWB Methyl parathion hydrolase OS = Pseudomonas sp.
(strain WBC-3) OX = 165468 GN = mpd PE = 1 SV = 1
(SEQ ID NO: 4)
(M)PLKNRLLARLSCVAAVVAATAAVAPLTLVSTAHAAAPQVRTSAPGYYRMLLGDFEITAL
SDGTVALPVDKRLNQPAPKTQSALAKSFQKAPLETSVTGYLVNTGSKLVLVDTGAAGLFG
PTLGRLAANLKAAGYQPEQVDEIYITHMHPDHVGGLMVGEQLAFPNAVVRADQKEADFWL
SQTNLDKAPDDESKGFFKGAMASLNPYVKAGKFKPFSGNTDLVPGIKALASHGHTPGHTT
YVVESQGQKLALLGDLILVAAVQFDDPSVTTQLDSDSKSVAVERKKAFADAAKGGYLIAA
SHLSFPGIGHIRAEGKGYRFVPVNYSVVNP
Sso-POX
Aryldialkylphosphatase
php
Saccharolobus solfataricus (strain ATCC 35092/DSM 1617/JCM 11322/P2)
(Sulfolobus solfataricus
>sp|Q97VT7|PHP_SACS2 Aryldialkylphosphatase OS = Saccharolobus solfataricus
(strain ATCC 35092/DSM 1617/JCM 11322/P2) OX = 273057 GN = php PE = 1 SV = 1
(SEQ ID NO: 5)
(M)RIPLVGKDSIESKDIGFTLIHEHLRVFSEAVRQQWPHLYNEDEEFRNAVNEVKRAMQFG
VKTIVDPTVMGLGRDIRFMEKVVKATGINLVAGTGIYIYIDLPFYFLNRSIDEIADLFIH
DIKEGIQGTLNKAGFVKIAADEPGITKDVEKVIRAAAIANKETKVPIITHSNAHNNTGLE
QQRILTEEGVDPGKILIGHLGDTDNIDYIKKIADKGSFIGLDRYGLDLFLPVDKRNETTL
RLIKDGYSDKIMISHDYCCTIDWGTAKPEYKPKLAPRWSITLIFEDTIPFLKRNGVNEEV
IATIFKENPKKFFS

In one embodiment, the protein comprises an amino acid sequence at least 75%, 80%, 85%, 90%, 91%, 92%, 93%, 94%, 95%, 96%, 97%, 98%, 99%, or 100% identical to the amino acid sequence of SEQ ID NO:1.

The proteins may further comprise a domain(s) that facilitates microbial cell surface expression of the protein. Any added domain that facilitates cell surface expression may be used as deemed appropriate for an intended use. In various non-limiting embodiments, the added domain(s) may comprise the INPNC ice-nucleation sequence (see, for example, U.S. Pat. No. 8,759,028, incorporated by reference herein in its entirety), or OmpA. In various embodiments, the added domain comprises an amino acid sequence at least 75%, 80%, 85%, 90%, 91%, 92%, 93%, 94%, 95%, 96%, 97%, 98%, 99%, or 100% identical to the amino acid sequence of SEQ ID NO: 6 or 7, wherein residues in parentheses are optional and may be present or absent, and when present may be substituted with any other amino acid linker of the same or different length and amino acid composition.

INPNC
(SEQ ID NO: 6)
(MA)LDKALVLRTCANNMADHCGLIWPASGTVESRYWQSTRRHENGLVGLLWGAGTSAFLSVHADARW
IVCEVAVADIISLEEPGMVKFPRAEVVHVGDRISASHFISARQADPASTSTSTSTSTLTPMPTAIPTP
MPAVASVTLPVAEQARHEVEDVASVSAAAAPVNTLPVTTPQNLQT(RS)RLWDGKRYRQLVARTGENG
VEADIPYYVNEDDDIVDKPDEDDDWIEVK
Lpp-OmpA
(SEQ ID NO: 7)
KATKLVLGAVILGSTLLAGCSSNAKIDQGINNNGPTHENQLGAGAFGGYQVNPYVGFEMGYDWLGRMPYKGSVEN
GAYKAQGVQLTAKLGYPITDDLDIYTRLGGMVWRADTKSNVYGKNHDTGVSPVFAGGVEYAITPEIATRPW

The non-native protein my include amino acid linkers connecting the protein and any domain(s) that facilitates microbial cell surface expression of the protein.

In one embodiment, the protein comprises an amino acid sequence at least 75%, 80%, 85%, 90%, 91%, 92%, 93%, 94%, 95%, 96%, 97%, 98%, 99%, or 100% identical to the amino acid sequence of SEQ ID NO:2, wherein the amino acid residues in parentheses are optional and may be present or absent, and when present may be substituted with any other amino acid linker of the same or different length and amino acid composition.

(SEQ ID NO: 2)
(MA)LDKALVLRTCANNMADHCGLIWPASGTVESRYWQSTRRHENGLVGLLWGAGTSAFLSVHADARW
IVCEVAVADIISLEEPGMVKFPRAEVVHVGDRISASHFISARQADPASTSTSTSTSTLTPMPTAIPTP
MPAVASVTLPVAEQARHEVFDVASVSAAAAPVNTLPVTTPQNLQT(RS)RLWDGKRYRQLVARTGENG
VEADIPYYVNEDDDIVDKPDEDDDWIEVK(SSNNNNNNNNNNLGASGSG)QTRRVVLKSAAAAGTLLG
GLAGCASVAGSIGTGDRINTVRGPITISEAGFTLTHEHICGSSAGELRAWPEFFGSRKALAEKAVRGL
RRARAAGVRTIVDVSTFDIGRDVSLLAEVSRAADVHIVAATGLWEDPPLSMRLRSVEELTQFFLREIQ
YGIEDTGIRAGIIKVATTGKATPFQELVLKAAARASLATGVPVTTHTAASQRDGEQQAAIFESEGLSP
SRVCIGHSDDTDDLSYLTALAARGYLIGLDHIPHSAIGLEDNASASALLGIRSWQTRALLIKALIDQG
YMKQILVSNDWLFGFSSYVTNIMDVMDRVNPDGMAFIPLRVIPFLREKGVPQETLAGITVINPARELS
PTLRAS

Bold: INPN , Bold and underlined: INPC and Unbolded: OPH

In embodiments wherein the protein comprises OPH, a degradation product generated by use of the microbial cells is p-nitrophenol (p-NP), which is both UV absorbent and electroactive.

Table 1 shows various non-limiting embodiments of organophosphate targets, exemplary enzymes that can be displayed on the recombinant microbial cell to degrade the OP target, and exemplary degradation products that can be generated using the enzyme.

TABLE 1

| | | Degradation |
|---|---|---|
| OP target | Enzyme | product |
| Parathion | OPH | p-NP |
| paraoxon | OPH | p-NP |
| paraoxon-methyl | OPH | p-NP |
| sarin | phosphotriesterase | F- |
| Cyclosarin | phosphotriesterase | F- |
| malathion | OPH | Diethyl fumarate |
| diazinon | OPH | IMP |
| Dichlorvos | Multi-enzyme | 2,2- |
| | | dichloropropanoic |
| | | acid, methanol, |
| | | 2,2,2- |
| | | trichloroethanol |
| chlorpyrifos | OPH | TCP |
| tetrachlorvinphos | | |
| oxydemeton methyl | Multi-enzyme | Bis[2- |
| | | (ehylthio)ethyl] |
| | | disulfide |
| phosmet | OPH | Pthalic anhydride |
| Soman | phosphotriesterase | F- |
| Tabun | phosphotriesterase | F- |
| VX | phosphotriesterase | F- |

The non-native protein is encoded by a nucleic acid in the microbial cell. In one embodiment, the nucleic acid may be present in a plasmid in the microbial cell, under the control of any promoter capable of promoting expression of the protein in the cell.

The recombinant microbial cell may be any bacterial, archaean, protozoan, or algal species capable of displaying on its surface the non-native protein capable of degrading an organophosphate. In one embodiment, the microbial cell is a bacteria. The bacteria may be any bacteria as deemed suitable for an intended use. In another embodiment, the bacteria is E. coli.

In one such embodiment, the cell is E. coli displaying an enzymatically active portion of parathion hydrolase from Pseudomonas diminuta on its surface. In another embodiment, the cell displays an enzymatically active portion of the amino acid sequence of SEQ ID NO:1 on its surface. In a further embodiment, the cell displays an enzymatically active portion of the amino acid sequence of SEQ ID NO:2 on its surface. In one such embodiment, the cell is lyophilized.

In a second aspect, the disclosure provides recombinant microbial cells engineered to be capable of expressing a non-native transcription factor that activates a non-native promoter in response to an organophosphate degradation product; wherein the non-native promoter is operatively linked to a nucleic acid encoding a reporter protein, wherein activity of the reporter protein can be detected. The recombinant microbial cell of this second aspect of the disclosure can be used, for example, in a co-culture organophosphate (“OP”) degradation product system with the microbial cells of the first aspect of the disclosure. Using such an engineered microbial system, the inventors have demonstrated detection of OP degradation products at parts per million levels, outperforming reported absorbance and fluorescence sensors.

The non-native transcription factor may be any transcription factor not normally expressed by the bacterial cell and which is activated (i.e.: actives transcription of a target promoter(s)) by an organophosphate degradation product of interest, including but not limited to those listed in Table 1. In one non-limiting embodiment, the phenol-responsive transcription factor, DmpR (dimethyl phenol regulator) from Pseudomonas spp. CF600, is known to undergo a conformational change upon interaction with a phenol such as p-NP. In other embodiments, the transcription factor can be any small molecule responsive transcription factor, such as a transcription factor that is responsive to an OP degradation product. Non-limiting embodiments include PPARγ (see, for example, Uniprot P37231) and acetate operon repressor (see, for example, Uniprot P16528). In another embodiment, the Nrf2 transcription factor may be used, which is responsive to dimethyl fumarate.

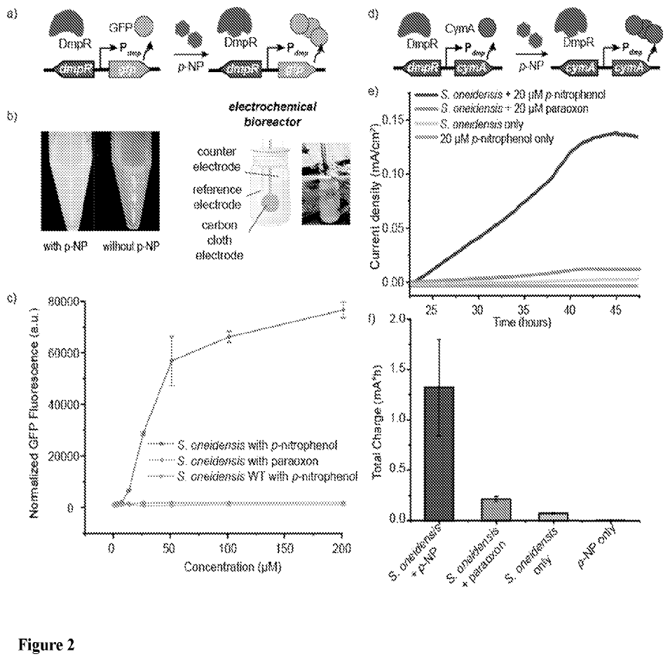

In one embodiment, the non-native transcription factor comprises DmpR, or variants thereof. See, for example, Wise and Kuske, Appl. Environ. Microbiol. 2000, 66 (1), 163-169, and FIG. 2 for variants; incorporated by reference herein in its entirety. In one embodiment, the non-native transcription factor comprises an amino acid sequence at least 75%, 80%, 85%, 90%, 91%, 92%, 93%, 94%, 95%, 96%, 97%, 98%, 99%, or 100% identical to the amino acid sequence of SEQ ID NO: 8, optionally modified with mutations Q10R and K117M, wherein residues in parentheses are optional and may be present or absent.

>tr|Q06573|Q06573_PSEUF DmpR protein OS = Pseudomonas sp. (strain CF600)
OX = 79676 GN = dmpR PE= 4 SV = 2
(SEQ ID NO: 8)
(M)PIKYKPEIQHSDFKDLTNLIHFQSMEGKIWLGEQRMLLLQFSAMASFRREMVNTLGIER
AKGLFLRHGYQSGLKDAELARKLRPNASEVGMFLAGPQMHSLKGLVKVRPTELDIDKEYG
RFYAEMEWIDSFEVEICQTDLGQMQDPVCWTLLGYACAYSSAFMGREIIFKEVSCRGCGG
DKCRVIGKPAEEWDDVASFKQYFKNDPIIEELYELQSQLLSLRTNLDKQEGQYYGIGQTP
AYQTVRNMMDKAAQGKVSVLLLGETGVGKEVIARSVHLRSKRAAEPFVAVNCAAIPPDLI
ESELFGVEKGAFTGATQSRMGRFERADKGTIFLDEVIELSPRAQASLLRVLQEGELERVG
DNRTRKIDVRVIAATHEDLAEAVKAGRFRADLYYRLNVFPVAIPALRERREDIPLLVEHF
LQRFHQEYGKRTLGLSDKALEACLHYSWPGNIRELENVIERGIILTDPNESISVQALFLR
APEEPQTASERVSLDGVLIQPGNGQGSWISQLLSSGLSLDEIEESLMREAMQQANQNVSG
AARLLGLSRPALAYRLKKIGIEG

The non-native promoter may be any promoter factor not normally expressed by the bacterial cell and which is activated (i.e., promotes expression of genes under its control) by the non-native transcription factor. In one non-limiting embodiment, the non-native promoter comprises Pdmp. In one embodiment, the non-native promoter comprises Pdmp obtained from the iGEM Registry of Standard Biological Parts. In one non-limiting embodiment, the promoter comprises the nucleic acid sequence of SEQ ID NO: 14.
Po promoter:

(SEQ ID NO: 14)
TAAGCATTTGCTCAAGCGGCCTTGGGCAATTGATCAAATGCTTAAAAAG
TCTGCGCAAGCGCGGCTTAATTTCGCTCGCTCCGATCATTCTAAAAATT
AGAAACACATTGAAAAACATTACCTTGAAGTCTGTTTTCAGACCTTGGC
ACAGCCGTTGCTTGATGTCCTGCGTACTAGAGTCACACAGGAAACCACT
AGA

In another embodiment, the promoter and transcription factor encoding nucleic acid sequence comprises the nucleic acid sequence of SEQ ID NO: 15.
Po promoter: Uppercase at 5′ end
PlmJ ribozyme sequence: Lower case and underlined immediately following promoter
Ribosome binding site sRBS4_cymA: Upper case
CymA coding sequence” Lowercase
L3S2P55 terminator: Lower case and underlined at 3′ end

(SEQ ID NO: 15)
5′
TAAGCATTTGCTCAAGCGGCCTTGGGCAATTGATCAAATGCTTAAAAAGTCTGCG
CAAGCGCGGCTTAATTTCGCTCGCTCCGATCATTCTAAAAATTAGAAACACATTG
AAAAACATTACCTTGAAGTCTGTTTTCAGACCTTGGCACAGCCGTTGCTTGATGTC
CTGCGTACTAGAGTCACACAGGAAACCACTAGAagtcataagtctgggctaagcccactgatgagtcg
ctgaaatgcgacgaaacttatgacctctacaaataattttgtttaaTTCGCTTTGGGTTTTTAAGGAGGACGCAa
tgaactggcgtgcactatttaaacccagcgcgaaatattccatcctagcgctactggttgttggtatcgtgattggtgttgtgggctattttg
caactcagcagactttacatgcgacaagtacagatgcgttctgtatgtcttgccatagcaatcattccttgaagaatgaagtgctggcatct
gcccacggtggcggcaaagccggggttactgttcagtgtcaagactgtcacttaccccatggccctgttgattatttaattaagaaaatca
tcgtatctaaagatttatatggtttcttaactattgatggctttaacactcaagcttggttagacgaaaaccgcaaagagcaagccgacaaa
gcattggcttacttccgtggtaacgactcagcaaactgtcaacactgccatactcgcatttatgaaaaccagccagaaaccatgaagcca
atggctgtgagaatgcacaccaacaacttcaagaaagatcctgaaacgagaaagacctgtgtggattgccacaaaggtgtcgctcacc
cctatccaaaaggataactcggtaccaaagacgaacaataagacgctgaaaagcgtcttttttcgttttggtcc 3′

The non-native transcription factor is encoded by a nucleic acid, such as in a plasmid in the microbial cell, under the control of any promoter capable of promoting expression of the transcription factor in the cell. The nucleic acid encoding the transcription factor and non-native promoter may be present in the same plasmid or different plasmids. In another embodiment, a nucleic acid encoding the non-native transcription factor is operatively linked to a constitutively active promoter.

The reporter protein may be any reporter protein with an activity that can be detected. In one embodiment, the reporter protein is any suitable fluorescent protein. In this embodiment, the microbial cell may be any bacterial, archaean, protozoan, or algal species.

In another embodiment, the reporter protein may be a protein component of an extracellular electron transfer (EET) system, including but not limited to periplasmic EET proteins Cytochrome c-type protein (CymA), Extracellular iron oxide respiratory system outer membrane component MtrA (MtrA), Extracellular iron oxide respiratory system outer membrane component MtrB (MtrB), Extracellular iron oxide respiratory system surface decaheme cytochrome c component MtrC (MtrC), Fumarate reductase flavoprotein subunit (FccA), or a functional fragment thereof. When the recombinant microbial cell of this aspect comprises Shewanella, the EET system comprises the “Mtr pathway”. In some embodiments, the reporter protein comprises an amino acid sequence at least 75%, 80%, 85%, 90%, 91%, 92%, 93%, 94%, 95%, 96%, 97%, 98%, 99%, or 100% identical to the amino acid sequence of any one of SEQ ID NO: 9-13, wherein residues in parentheses are optional and may be present or absent.

### Cytochrome c-type protein

### cymA

Shewanella oneidensis (strain MR-1)
>tr|Q8E8S0|Q8E8S0_SHEON Cytochrome c-type protein OS = Shewanella oneidensis
(strain MR-1) OX = 211586 GN = cymA PE = 3 SV = 1
(SEQ ID NO: 9)
(M)NWRALFKPSAKYSILALLVVGIVIGVVGYFATQQTLHATSTDAFCMSCHSNHSLKNEVL
ASAHGGGKAGVTVQCQDCHLPHGPVDYLIKKIIVSKDLYGFLTIDGENTQAWLDENRKEQ
ADKALAYFRGNDSANCQHCHTRIYENQPETMKPMAVRMHTNNFKKDPETRKTCVDCHKGV
AHPYPKG

Submitted name:

### Extracellular iron oxide respiratory system periplasmic decaheme cytochrome c component MtrA

### mtrA

Shewanella oneidensis (strain MR-1)
>tr|Q8EG35|Q8EG35_SHEON Extracelllular iron oxide respiratory system
periplasmic decaheme cytochrome c component MtrA OS = Shewanella oneidensis
(strain MR-1) OX = 211586 GN = mtrA PE = 1 SV = 1
(SEQ ID NO: 10)
(M)KNCLKMKNLLPALTITMAMSAVMALVVTPNAYASKWDEKMTPEQVEATLDKKFAEGNYS
PKGADSCLMCHKKSEKVMDLFKGVHGAIDSSKSPMAGLQCEACHGPLGQHNKGGNEPMIT
FGKQSTLSADKQNSVCMSCHQDDKRMSWNGGHHDNADVACASCHQVHVAKDPVLSKNTEM
EVCTSCHTKQKADMNKRSSHPLKWAQMTCSDCHNPHGSMTDSDLNKPSVNDTCYSCHAEK
RGPKLWEHAPVTENCVTCHNPHGSVNDGMLKTRAPQLCQQCHASDGHASNAYLGNTGLGS
NVGDNAFTGGRSCLNCHSQVHGSNHPSGKLLQR

Submitted name:

### Extracellular iron oxide respiratory system outer membrane component MtrB

### mtrB

Shewanella oneidensis (strain MR-1)
>tr|Q8CVD4|Q8CVD4_SHEON Extracellular iron oxide respiratory system outer
membrane component MtrB OS = Shewanella oneidensis (strain MR-1)
OX = 211586 GN = mtrB PE = 1 SV = 1
(SEQ ID NO: 11)
(M)KFKLNLITLALLANTGLAVAADGYGLANANTEKVKLSAWSCKGCVVETGTSGTVGVGVG
YNSEEDIRSANAFGTSNEVAGKFDADLNFKGEKGYRASVDAYQLGMDGGRLDVNAGKQGQ
YNVNVNYRQIATYDSNSALSPYAGIGGNNLTLPDNWITAGSSNQMPLLMDSLNALELSLK
RERTGLGFEYQGESLWSTYVNYMREEKTGLKQASGSFFNQSMMLAEPVDYTTDTIEAGVK
LKGDRWFTALSYNGSIFKNEYNQLDFENAFNPTFGAQTQGTMALDPDNQSHTVSLMGQYN
DGSNALSGRILTGQMSQDQALVTDNYRYANQLNTDAVDAKVDLLGMNLKVVSKVSNDLRL
TGSYDYYDRDNNTQVEEWTQISINNVNGKVAYNTPYDNRTQRFKVAADYRITRDIKLDGG
YDFKRDQRDYQDRETTDENTVWARLRVNSFDTWDMWVKGSYGNRDGSQYQASEWTSSETN
SLLRKYNLADRDRTQVEARITHSPLESLTIDVGARYALDDYTDTVIGLTESKDTSYDANI
SYMITADLLATAFYNYQTIESEQAGSSNYSTPTWTGFIEDQVDVVGAGISYNNLLENKLR
LGLDYTYSNSDSNTQVRQGITGDYGDYFAKVHNINLYAQYQATEKLALRFDYKIENYKDN
DAANDIAVDGIWNVVGFGSNSHDYTAQMLMLSMSYKL

Submitted name:

### Extracellular iron oxide respiratory system surface decaheme cytochrome c component MtrC

### mtrC

Shewanella oneidensis (strain MR-1)
>tr|Q8EG34|Q8EG34_SHEON Extracellular iron oxide respiratory system surface
decaheme cytochrome c component MtrC OS = Shewanella oneidensis (strain MR-1)
OX = 211586 GN = mtrC PE = 1 SV = 1
(SEQ ID NO: 12)
MMNAQKSKIALLLAASAVTMALTGCGGSDGNNGNDGSDGGEPAGSIQTLNLDITKVSYEN
GAPMVTVFATNEADMPVIGLANLEIKKALQLIPEGATGPGNSANWQGLGSSKSYVDNKNG
SYTFKFDAFDSNKVFNAQLTQRFNVVSAAGKLADGTTVPVAEMVEDFDGQGNAPQYTKNI
VSHEVCASCHVEGEKIYHQATEVETCISCHTQEFADGRGKPHVAFSHLIHNVHNANKAWG
KDNKIPTVAQNIVQDNCQVCHVESDMLTEAKNWSRIPTMEVCSSCHVDIDFAAGKGHSQQ
LDNSNCIACHNSDWTAELHTAKTTATKNLINQYGIETTSTINTETKAATISVQVVDANGT
AVDLKTILPKVQRLEIITNVGPNNATLGYSGKDSIFAIKNGALDPKATINDAGKLVYTTT
KDLKLGQNGADSDTAFSFVGWSMCSSEGKFVDCADPAFDGVDVTKYTGMKADLAFATLSG
KAPSTRHVDSVNMTACANCHTAEFEIHKGKQHAGFVMTEQLSHTQDANGKAIVGLDACVT
CHTPDGTYSFANRGALELKLHKKHVEDAYGLIGGNCASCHSDENLESFKKKGALNTAAAA
DKTGLYSTPITATCTTCHTVGSQYMVHTKETLESFGAVVDGTKDDATSAAQSETCFYCHT
PTVADHTKVKM

### Fumarate reductase flavoprotein subunit

### fccA

Shewanella frigidimarin
>sp|P0C278|FRDA_SHEFR Fumarate reductase flavoprotein subunit OS = Shewanella
frigidimarina OX = 56812 GN = fccA PE = 1 SV = 1
(SEQ ID NO: 13)
ADNLAEFHVQNQECDSCHTPDGELSNDSLTYENTQCVSCHGTLEEVAETTKHEHYNAHAS
HFPGEVACTSCHSAHEKSMVYCDSCHSFDENMPYAKKWQRDEPTIAELAKDKSERQAALA
SAPHDTVDVVVVGSGGAGFSAAISATDSGAKVILIEKEPVIGGNAKLAAGGMNAAWTDQQ
KAKKITDSPELMFEDTMKGGQNINDPALVKVLSSHSKDSVDWMTAMGADLTDVGMMGGAS
VNRAHRPTGGAGVGAHVVQVLYDNAVKRNIDLRMNTRGIEVLKDDKGTVKGILVKGMYKG
YYWVKADAVILATGGFAKNNERVAKLDPSLKGFISTNQPGAVGDGLDVAENAGGALKDMQ
YIQAHPTLSVKGGVMVTEAVRGNGAILVNREGKRFVNEITTRDKASAAILAQTGKSAYLI
FDDSVRKSLSKIDKYIGLGVAPTADSLVKLGKMEGIDGKALTETVARYNSLVSSGKDTDE
ERPNLPRALNEGNYYAIEVTPGVHHTMGGVMIDTKAEVMNAKKQVIPGLYGAGEVTGGVH
GANRLGGNAISDIITFGRLAGEEAAKYSKKN

In these embodiments, the microbial cell may be any bacterial, archaean, protozoan, or algal species that is either naturally or genetically engineered to perform EET.

In one embodiment, the microbial cell is a bacteria. In another embodiment, wherein the microbial cell is any Shewanella including but not limited to S. oneidensis, or any species of Geobacter. In one embodiment, the cell is S. oneidensis comprising a nucleic acid encoding non-native transcription factor DmpR comprising the amino acid sequence of SEQ ID NO: 8, or a modified version thereof (such as modified with mutations Q10R and K117M) operatively linked to a constitutive promoter; wherein the reporter protein comprises an amino acid sequence comprising the amino acid sequence of SEQ ID NO: 9 (CymA) or a functional fragment thereof.

In a third aspect, the disclosure provides kits, comprising
(a) a first recombinant microbial cell comprising the recombinant microbial cell of any embodiment or combination of embodiments of the first aspect of the disclosure; and
(b) a second recombinant microbial cell comprising the recombinant microbial cell of any embodiment or combination of embodiments of the second aspect of the disclosure.

The kits of the disclosure may be used, for example, as a co-culture organophosphate (“OP”) degradation product system. Using such an engineered microbial system, the inventors have demonstrated (as provided in the attached examples) detection of OP degradation products at parts per million levels, outperforming reported absorbance and fluorescence sensors.

All embodiments of the first aspect and second aspects described above may be used in any combination in the kits of the disclosure as deemed suitable for an intended purpose.

In one specific embodiment:
(a) the first recombinant microbial cell comprises E. coli displaying an enzymatically active portion of parathion hydrolase from Pseudomonas diminuta on its surface; and
(b) the second recombinant microbial cell comprises S. oneidensis comprising a nucleic acid encoding non-native transcription factor DmpR comprising the amino acid sequence of SEQ ID NO: 8, or a modified version thereof (such as modified with mutations Q10R and K117M) operatively linked to a constitutive promoter; wherein the reporter protein comprises an amino acid sequence comprising the amino acid sequence of SEQ ID NO: 9 (CymA) or a functional fragment thereof.

In a fourth aspect, the disclosure provides methods for degrading organophosphates, comprising contacting a source suspected of containing organophosphates with a first recombinant microbial cell of embodiment or combination of embodiments of the first aspect of the disclosure, thus degrading organophosphates present in the source.

In another embodiment, the methods comprise methods for degradation of organophosphates and detection of organophosphate degradation products, comprising
(a) contacting a source suspected of containing organophosphates with (i) a first recombinant microbial cell of embodiment or combination of embodiments of the first aspect of the disclosure, thereby degrading organophosphates present in the source to generate organophosphate degradation products; and (ii) a second recombinant microbial cell of any embodiment or combination of embodiments of the second aspect of the disclosure;
(b) detecting reporter protein signal, wherein the reporter protein signal provides a measure of organophosphate degradation products in the source.

The inventors have demonstrated (as provided in the attached examples) that the methods of the disclosure generate OP degradation products and provide for detection of such OP degradation products at parts per million levels, outperforming reported absorbance and fluorescence sensors. All embodiments of the first, second, and third aspects described above may be used in any combination of the methods of the disclosure as deemed suitable for an intended purpose. When the reporter comprises a fluorescent reporter protein, detection methods may be any fluorescence detection methods as deemed suitable for an intended use, including but not limited to those disclosed in the examples. Similarly, when the reporter comprises a component of an EET pathway, detection methods may be any electrochemical detection method capable of detecting electrical current generated by the expression of the reporter protein in EET-competent cells as deemed suitable for an intended use, including but not limited to those disclosed in the examples.

In one embodiment, the recombinant microbial cell is wherein the cell is E. coli displaying an enzymatically active portion of parathion hydrolase from Pseudomonas diminuta on its surface, wherein contacting the source with the recombinant E. coli degrades organophosphates present in the source and generates p-Np degradation products. In another embodiment, the cell displays an enzymatically active portion of the amino acid sequence of SEQ ID NO:1 or SEQ ID NO:2 on its surface. In another embodiment, the cell is lyophilized.

In a further embodiment, the second recombinant microbial cell is S. oneidensis comprising a nucleic acid encoding non-native transcription factor DmpR having the amino acid sequence provided at Uniprot ID: Q06573 or a modified version thereof (such as modified with mutations Q10R and K117M) operatively linked to a constitutive promoter; wherein the non-native promoter comprises Pdmp operatively linked to CymA or a functional fragment thereof; and the reporter protein signal comprises an electrochemical response, wherein an increase in current relative to control provides a measure of p-Np in the source.

The source may be any source in which OP may be present. In various non-limiting embodiments, the source comprises water of any type including but not limited to lakes streams, rivers, well water, groundwater, drinking water, and agricultural runoff.

### EXAMPLES

Organophosphate (OP) pesticides cause hundreds of illnesses and deaths annually. Unfortunately, exposures are often detected by monitoring degradation products in blood and urine, with few effective methods for detection and remediation at the point of dispersal. We have developed an innovative strategy to remediate these compounds: an engineered microbial technology for the targeted detection and destruction of OP pesticides. This system is based upon microbial electrochemistry using two engineered strains. The strains are combined such that the first microbe (E. coli) degrades the pesticide, while the second (S. oneidensis) generates current in response to the degradation product without requiring external electrochemical stimulus or labels. This cellular technology is unique in that the E. coli serve only as an inert scaffold for enzymes to degrade OPs, circumventing a fundamental requirement of co-culture design: maintaining the viability of two microbial strains simultaneously. With this platform, we can detect OP degradation products at sub-micromolar levels, outperforming reported colorimetric and fluorescence sensors. Importantly, this approach affords a modular, adaptable strategy that can be expanded to additional environmental contaminants.

### Introduction

The organophosphate (OP) pesticides parathion and paraoxon cause thousands of illnesses and deaths annually, as they have the same mechanism of action as the nerve gas sarin. Such compounds disrupt the native neurotransmitter acetylcholine and impact the parasympathetic nerve system, which can be deadly. Similar compounds were employed as chemical weapons during World War II in the form of sarin, cyclosarin, and soman. OP chemical warfare agents (CWAs) have continued to be used as recently as 2017. However, the main application of OPs is not as CWAs but as insecticides and pesticides. In fact, OPs are the most widely-used pesticides in industrialized countries, causing environmental contamination and posing significant danger following exposure. Annually, there are an estimated three million exposures to OPs, causing 300,000 fatalities. The continued danger of these pesticides necessitates vigilance and affordable, easy-to-use technologies for detection and remediation, especially in low resource settings.

Here, we report an engineered dual species technology for the targeted detection and destruction of parathion-type OPs. This technology is based on electrochemical signals generated by electroactive microbes in response to OP degradation products. Electroactive microbes generate through extracellular electron transfer (EET) by supplying their metabolic electrons to an external electron acceptor, such as a poised electrode. S. oneidensis and similar microbes use multiheme cytochrome protein conduits spanning from the inner to the outer membrane and diffusible redox mediators to directly or indirectly perform EET. The first microbe in this technology, E. coli, degrades the pesticide, while the second (S. oneidensis) generates current in response to the degradation product without external electrochemical stimulus or labels. This cell mixture is unique in that enzymes displayed on E. coli function even when the cells are lyophilized, enabling the cells to serve only as an inert scaffold and circumventing a core challenge in co-culture design: maintaining the viability of two microbial strains simultaneously. Using this engineered microbial system, we have demonstrated the detection of OP degradation products at parts per million levels, outperforming reported absorbance and fluorescence sensors.⁶⁵ Our approach affords a modular, adaptable strategy through dual microbial engineering to control electron transfer in one organism with molecules generated from contaminant degradation by another.

### Results

### Degradation of Organophosphates Using Engineered E. coli

We have used the surface of E. coli as a platform to display organophosphate hydrolase (OPH) for OP degradation. The OPH used in this study is a phosphotriesterase obtained from Pseudomonas diminuta. Also known as parathion hydrolase, the enzyme specifically degrades synthetic OP triesters and phosphorofluoridates with high catalytic efficiency. Using our previously-reported strategy employing the INPNC ice-nucleation sequence, OPH was displayed on the cell surface.⁶⁴ The ice-nucleation sequence circumvents the cytotoxic effects of the more common surface expression tag, OmpA. Moreover, the cells are lyophilized following OPH expression, making their viability unnecessary for OP degradation and potentially increasing the length of their storage life—both important for an optimal deployable technology. Extrapolating from previous INPNC E. coli expression, we estimate approximately 50,000 enzymes to be expressed per lyophilized cell using our induction conditions.

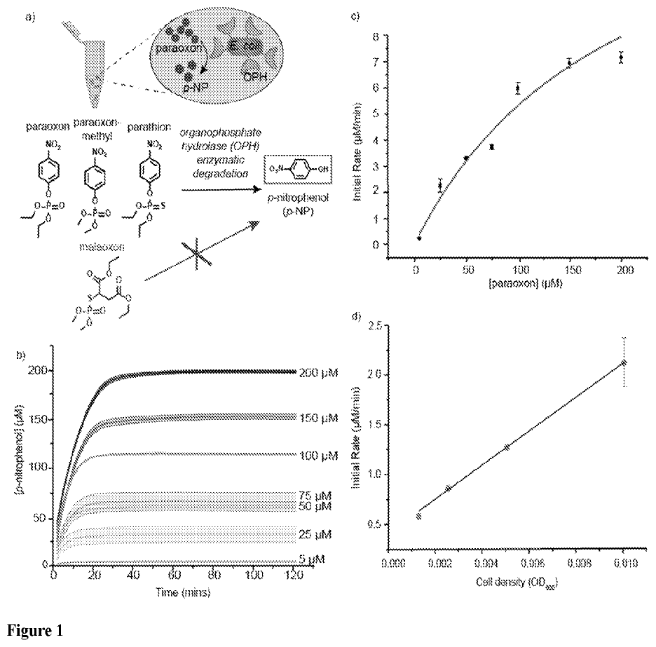

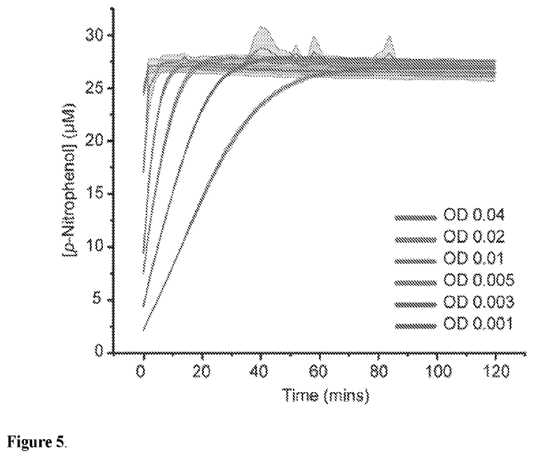

The degradation of paraoxon by lyophilized OPH-E. coli was confirmed by monitoring the OP degradation product, p-NP, using a colorimetric assay (FIG. 1). Under alkaline conditions, p-NP forms a phenolate ion, which is yellow in color and has a maximum absorbance at 400 nm. The absorbance values obtained from the colorimetric assay were converted to p-NP concentrations with the help of a standard curve. We can therefore quantify paraoxon degradation through the production of p-NP at a fixed cell density (OD₆₀₀ 0.02) (FIG. 1b). At each paraoxon concentration, rapid initial p-NP production followed by equilibration was observed. From these data, enzyme kinetic parameters were determined by fitting the Michaelis-Menten equation (FIG. 1c). The Michaelis constant (K_{M}) of paraoxon for the enzyme was calculated to be 197.9±81.7 μM, which is slightly higher than the reported affinity of paraoxon for purified OPH.⁶⁶ This is hypothesized to be due to restricted substrate diffusion to the enzymes that are confined on the cells rather than free in solution. To determine the effect of enzyme concentration on substrate turnover, a fixed concentration of paraoxon was added to varying concentrations of lyophilized OPH-E. coli (FIG. 5). As anticipated, the initial reaction rate of paraoxon degradation is directly proportional to the cell concentration and therefore, the OPH enzymes expressed on the E. coli cell surface (FIG. 1d). These results indicate that the OPH enzymes on the surface of E. coli retain their biologically-relevant conformation even following lyophilization. The degradation of paraoxon, therefore, does not require viable E. coli to proceed.

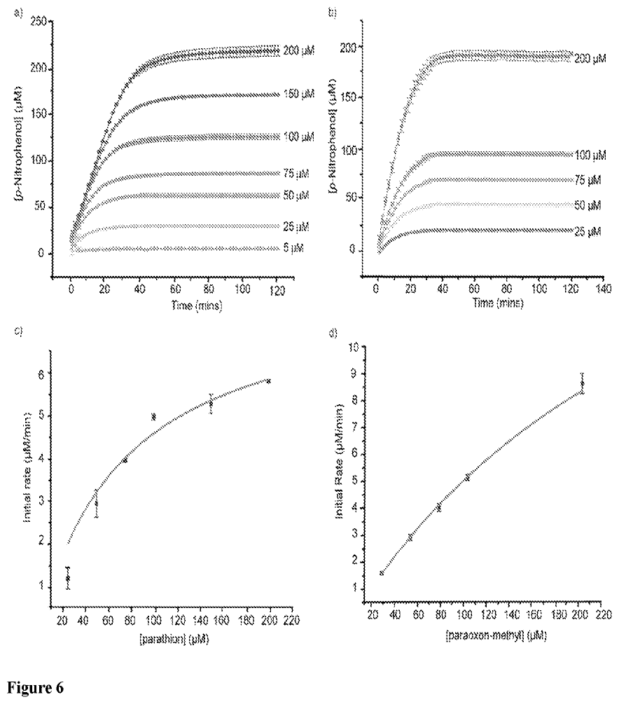

To evaluate the generalizability of this biomaterial for OP remediation, we tested additional organophosphates. Parathion is another especially common OP pesticide, but it contains a phosphorus-sulfur bond in place of the phosphorus-oxygen bond found in paraoxon. We evaluated the ability of our OPH-E. coli to degrade parathion and observed similar results to paraoxon degradation (FIG. 6a, c). The catalytic efficiency (Vₘₐₓ/K_{M}) is 0.079±0.13 min⁻¹ for paraoxon and 0.104 min⁻¹±0.013 for parathion. We further evaluated the degradation of paraoxon-methyl, which differs from paraoxon in the substituents on the phosphate. Under the same experimental conditions, very slow rates of p-NP production were observed, and the rate did not reach equilibrium even after two hours. Upon reducing the concentration of the solvent methanol from 0.1% to 0.01%, significantly enhanced degradation rates for paraoxon-methyl were observed (FIG. 6b,d), indicating enzyme inhibition by methanol. Polar solvents have been suggested to competitively inhibit the OPH active site. Taken in total, our results demonstrate that the OPH expressed on E. coli can degrade a variety of OPs, albeit with differing efficiencies.

### Fluorescent Detection of the Organophosphate Degradation Product p-NP by Engineered S. oneidensis

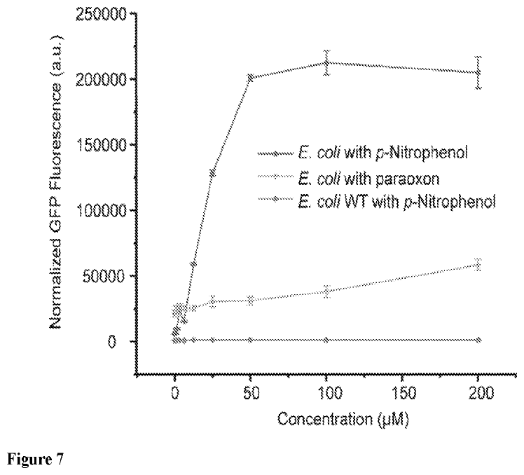

Electrochemical sensors to detect OP pesticides most often rely on the direct detection of p-NP. However, off-target phenols that are common in environmental matrices can have similar electrochemical properties, causing false positive results. To enhance the specificity and sensitivity of our biosensing system, we engineered the electroactive microbe S. oneidensis such that the expression of EET machinery, and hence current production, is triggered by p-NP. A phenol-responsive transcription factor, DmpR (dimethyl phenol regulator) from Pseudomonas spp. CF600, is known to undergo a conformational change upon interaction with a phenol such as p-NP, activating the DmpR promoter, P_{dmp}. To determine whether DmpR responds to p-NP, we initially placed the induction of green fluorescent protein (GFP) under the control of P_{dmp} in both S. oneidensis and E. coli (FIG. 2a), as GFP fluorescence facilitated high-throughput analysis. In the engineered S. oneidensis, a 54-fold increase in fluorescence was observed with 200 μM p-NP, with fluorescence observed at p-NP concentrations as low as 6.25 μM (FIG. 2b,c). Further, gene expression was found to be logarithmic, showing a sharp increase in fluorescence at 6.25 μM p-NP, which nearly levels off at concentrations higher than 50 M. No such increase in the fluorescence was observed for wildtype (WT) S. oneidensis following the addition of paraoxon. E. coli similarly displayed a logarithmic increase in GFP expression upon a p-NP concentration increase from 6.25 μM to 50 M (FIG. 7). This response was significantly higher than for WT E. coli treated with p-NP or GFP-expressing cells treated with paraoxon. These results confirm that the transcription factor, DmpR, in the engineered cells is responsive to the inducer p-NP. Further, this response is specific to the OP degradation product, not to the OP itself.

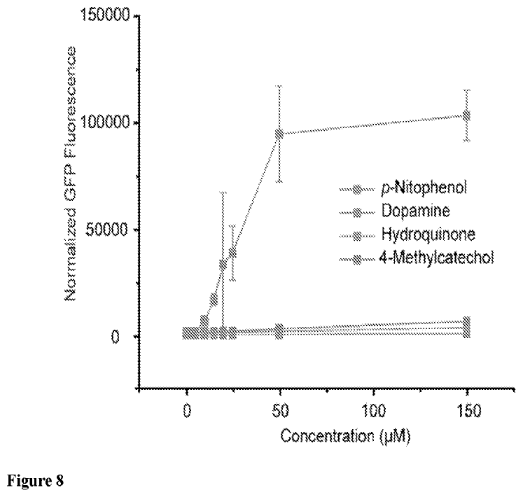

To confirm the specificity of DmpR for p-NP and not off-target phenols found in natural systems, we performed a GFP assay on off-target compounds. By exposing the engineered p-NP-responsive S. oneidensis to different concentrations of three environmentally-relevant off-target compounds (dopamine, hydroquinone, and 4-methylcatechol), the specificity of this transcription factor was confirmed. Compared to the normalized GFP fluorescence emissions of p-NP, the off-target compounds produced very low fluorescence, providing further evidence for the specificity of the sensor and its viability for field deployment (FIG. 8).

### Electrochemical Detection of p-NP by Engineered S. oneidensis

We next evaluated whether p-NP-activated DmpR can elicit an electrochemical response in S. oneidensis. Here, we placed expression of an inner membrane bound EET protein, CymA, under control of P_{dmp}. CymA is a tetraheme quinol dehydrogenase responsible for directing metabolic electron flux to the extracellular space through the MtrABC conduit. CymA was selected over other Mtr proteins (MtrB and MtrC), as we observed highest induction and minimal leakiness in the IPTG-inducible knockout strains that were used to construct the p-NP-inducible strains. Constitutively-expressed DmpR activates P_{dmp} in the presence of p-NP, leading to the expression of CymA (FIG. 2d), which “completes the circuit” and enables EET in S. oneidensis. We validated this response by introducing the engineered cells to a three-electrode bioreactor consisting of a carbon cloth working electrode poised at +400 mV vs. silver chloride/silver (AgCl/Ag) (FIG. 2c). Cells were provided with lactate as an electron donor and carbon source, and the poised carbon cloth electrode is expected to function as an electron acceptor only in the presence of p-NP. The reactor headspace was open to the atmosphere to allow O₂ diffusion for cells to use as an alternate electron acceptor in the absence of p-NP, and S. oneidensis can respire electrodes under low O₂ tension indicating that the presence of O₂ does not hinder EET by the cells. A continuous increase in anodic current, indicating electron transfer from cells to the electrode, was observed only following addition of p-NP to the bioreactor. Cells alone, p-NP alone, and cells with paraoxon did not yield a considerable current (FIG. 2e, 9). At 20 μM p-NP, the cells generated a total charge of 1.32±0.48 mA*h in 24 hours; the cells without p-NP added or with paraoxon added generated only 0.072±0.006 mA*h charge and 0.21±0.02 mA*h charge in the same amount of time, respectively (FIG. 2f). The small current observed in the presence of paraoxon is likely due to low levels of its non-enzymatic degradation. Taken together, these results confirm that p-NP specifically leads to CymA expression, allowing the cells to perform EET to an external electrode. Overall, our fluorescence and electrochemical measurements indicate that the transcription factor DmpR is activated by the OP degradation product p-NP, which in turn induces the expression of target proteins (either GFP or CymA).

### GFP Fluorescence Response of Engineered Co-cultures to Organophosphates

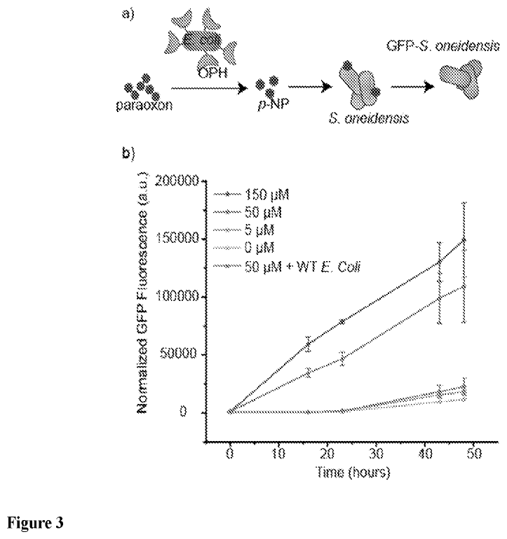

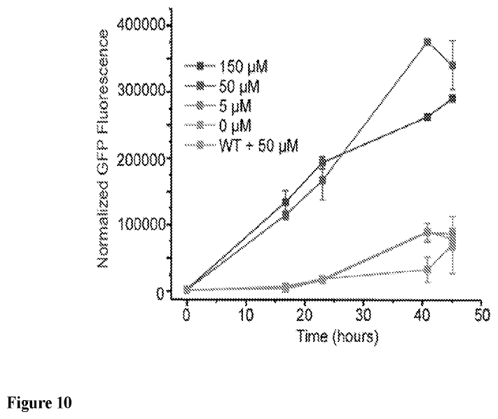

After independently confirming OP degradation activity by lyophilized OPH-E. coli and p-NP detection by engineered S. oneidensis in each monoculture, we assessed the activity of the two microbes together in the presence of OPs. For initial validation, we added lyophilized OPH-E. coli to DmpR-driven, GFP-expressing S. oneidensis. GFP fluorescence was monitored over time in the presence of paraoxon (FIG. 3a). We observed an increase in GFP fluorescence for cell mixtures with an initial paraoxon concentration of 5, 50 and 150 μM (FIG. 3b), and the magnitude of the fluorescence was dependent on the initial OP concentration. From the time of inoculation to 24 hours, a 55-fold increase in fluorescence was observed for co-cultures with 150 μM paraoxon added; a 35-fold increase was observed with 50 μM paraoxon, and a 2.5-fold increase was observed with 5 μM paraoxon. Minimal fluorescence was observed in co-cultures containing lyophilized WT E. coli in place of OPH-expressing E. coli. Similar degradation and detection efficiencies were observed in a dual E. coli system (OPH-E. coli and DmpR driven GFP expressing E. coli) (FIG. 10). These results, combined with monoculture experiments, suggest that OPH-E. coli-mediated paraoxon degradation activates DmpR in engineered S. oneidensis, leading to an increase in GFP fluorescence over time. Furthermore, our results indicate that GFP induction by p-NP in engineered S. oneidensis is concentration dependent, enabling a quantitative measure of OP concentration.

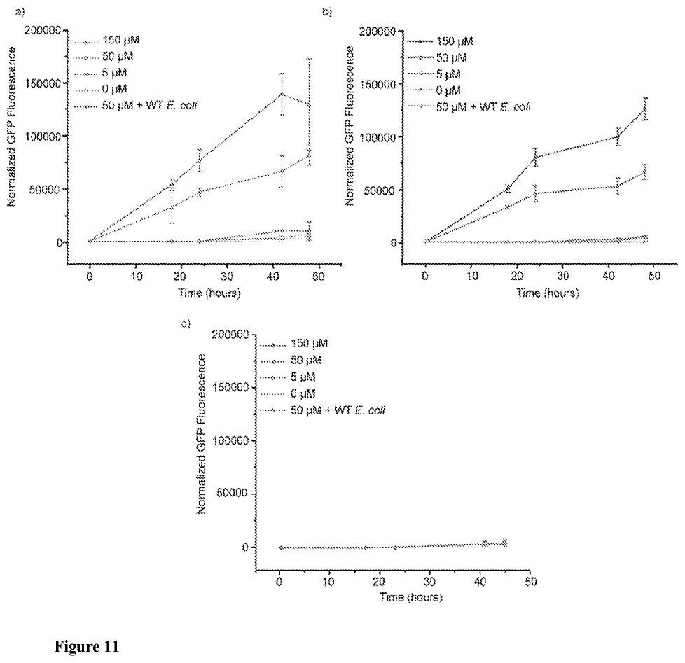

Based on the initial success of the mixture, we investigated whether other OPs (paraoxon-methyl, parathion, and malaoxon) generate a GFP response in our lyophilized OPH-E. coli and engineered S. oneidensis combination. We found similar degradation and detection efficiencies with paraoxon-methyl, parathion, and paraoxon (FIG. 11a,b). At 150 μM initial OP concentration, the mixtures with parathion generated a 58-fold increase in fluorescence, and those with paraoxon-methyl generated a 48-fold increase within 24 hours of inoculation. These results confirm that OPs that generate a p-NP degradation product elicit GFP expression in our engineered S. oneidensis. As a negative control, we evaluated malaoxon, which does not yield a phenolic degradation product. As predicted, GFP expression was not observed in our cell mixtures with malaoxon, further validating the specificity of the response (FIG. 11c). Overall, our results confirm that the increase in fluorescence in OPH-E. coli/S. oneidensis mixtures is due to the degradation of OPs to p-NP, leading to GFP expression in S. oneidensis. Furthermore, this system functions for any OP substrate that is hydrolyzed to p-NP.

### Electrochemical Response of Engineered Co-cultures to Organophosphates

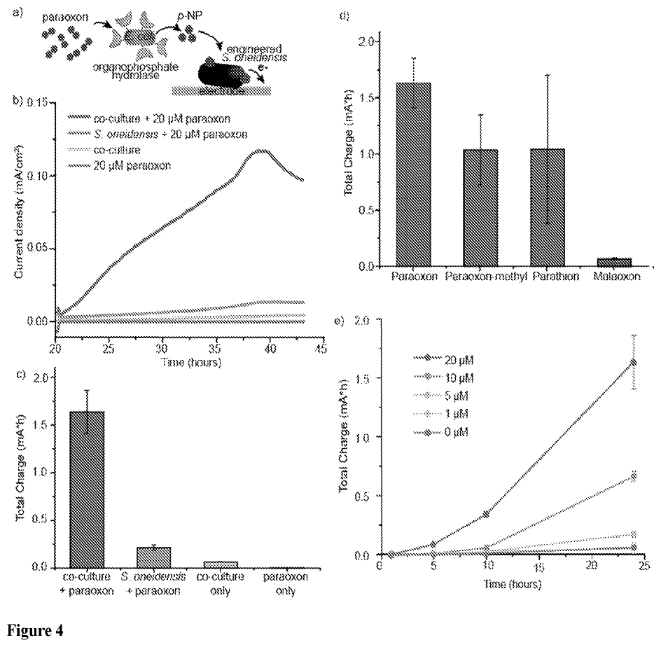

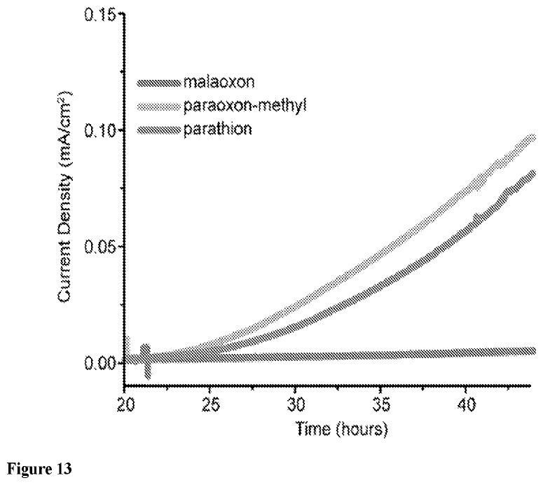

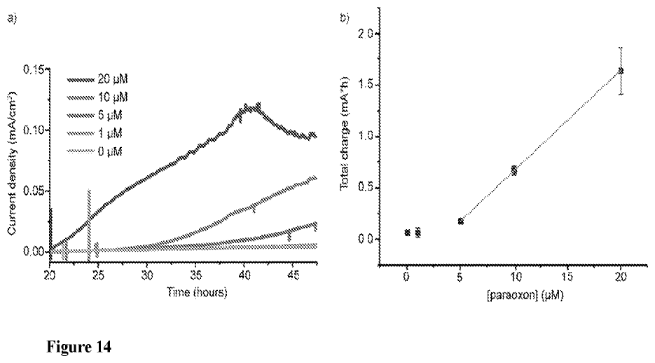

To generate an electrochemical biosensor for OP detection and degradation, we combined S. oneidensis that expresses the critical EET protein CymA in response to p-NP with lyophilized OPH-E. coli (FIG. 4a). The electrochemical response of engineered cell mixtures was measured in three-electrode bioreactors, identical to the monoculture experiments. An increase in anodic current was observed only following the addition of paraoxon to the bioreactor containing both lyophilized OPH-E. coli and engineered S. oneidensis (FIG. 4b, 12). This increase was significantly higher than the current produced by either the cell mixture in the absence of paraoxon or the paraoxon alone, which generated current similar to that of the media baseline. The slight decrease in current density in the co-culture reactors with paraoxon after 40 hours could likely be due to electron donor depletion and is often observed in S. oneidensis biorcactors. The mixture with 20 μM paraoxon accumulated a total charge of 1.64±0.18 mA*h in 24 hours. In comparison, the cell mixture alone accumulated only 0.065±0.002 mA*h of charge (FIG. 4c). These results, combined with the observation that S. oneidensis monocultures produce anodic current only in the presence of p-NP, indicate that OPH-E. coli-mediated paraoxon hydrolysis leads to CymA expression in S. oneidensis, thereby initiating EET to the carbon cloth electrode. In agreement with our fluorescence experiments, we observed paraoxon-methyl and parathion yielded an increase in anodic current with the engineered cell mixtures. On the other hand, no significant current is observed with malaoxon, which does not generate p-NP (FIG. 13). Comparable with paraoxon, in 24 hours, we observed 1.03±0.31 and 1.04±0.66 mA*h charge with paraoxon-methyl and parathion, respectively, but only 0.068±0.006 mA*h from malaoxon (FIG. 4d), which is equivalent to the co-culture only control. Our results consistently show that the current response is directly proportional to the initial concentration of paraoxon (FIG. 14a). The total charge produced by the bioreactors was calculated at 5, 10 and 24 hours (FIG. 4e). Within 5 hours, 20 μM paraoxon could be detected with charge accumulation of 0.140±0.02 mA*h. At 24 hours, the charge produced by the cell mixture with 5 μM paraoxon (0.180±0.018 mA*h) was approximately three times higher and with 1 μM concentration (0.101±0.017 mA*h) was 1.7 times higher than the cell only baseline. By determining the slope(S) and standard deviation of the intercept (Sy) from the linear fit of the calibration curve (FIG. 14b), the limit of detection of the sensor was calculated to be 0.15 μM (3.3×(Sy/S)). Though we did not explicitly evaluate current generation as a function of p-NP concentration, we have demonstrated current generation to be the rate-limiting step for the degradation of paraoxon and detection of p-NP. Thus, we expect similar results for the direct detection of p-NP to those observed with varying paraoxon concentrations, in which a linear range of detection is observed between 5 and 20 μM (FIG. 14). Our electrochemical measurements thus confirm that this technology is sensitive and specific. Further, it is capable of degradation and detection of toxic OPs in a single assay.

### Discussion

In this study, we have shown that engineered microbial combinations can both degrade and detect toxic OPs with incomparable specificity and sensitivity using electrochemical readout. E. coli were engineered to express OPH on the cell surface to degrade organophosphate pesticides to p-NP, even following E. coli lyophilization. We also engineered electroactive microbes, S. oneidensis, to express a critical EET protein, CymA, in the presence of p-NP. Expression of CymA completes the electron transfer pathway allowing the bacteria to respire external electrodes. After validating the function of the two engineered microbes individually, our co-culture studies confirm that OPH-expressing E. coli degrade OPs and that S. oneidensis detect the degradation product and generate current as a readout in a single assay.

The state-of-the-art for OP remediation and biosensing relies on assays that either degrade or detect the OPs. Using our engineered bacterial cell mixture, our electrochemical assay can perform both functions in a single system. OPH had previously been used to enzymatically degrade OPs, but these technologies suffer from OPH instability. Expression of this enzyme on the E. coli surface circumvents difficulties with protein handling. Further, our OPH-expressing E. coli are lyophilized and non-viable, yet they maintain their enzymatic degradation activity. This is an important development, as we have bypassed the need to develop optimal culture conditions for two microbes to maintain metabolic activity in co-culture. Lyophilization also allows for long-term storage of the enzyme-modified E. coli, making them highly suitable for field-based OP biosensing and remediation.

With our engineered S. oneidensis, the current is specific to the presence of p-NP, and thus the possibility of false positives is minimized. EET in the bacteria is “turned-on” only in its presence. By using whole cell-based assay with lyophilized bacteria, our biosensing strategy can be used as a cheap and robust system for field deployment. Overall, our engineered microbes have streamlined OP biodegradation and biosensing in a single system. This technology provides a significant improvement over the current biosensors in terms of handling, storage, and specificity.

Our electrochemical cell system enables continuous monitoring without the need for sampling or sample processing, as is required for fluorescence-based assays. The current study provides a proof-of-principle for the two engineered microbes to simultaneously degrade and detect dangerous, environmental contaminants. Improving the fitness of the transcription factor DmpR for enhanced interaction with p-NP, could result in faster p-NP detection and response times by S. oneidensis. Additionally, since this strategy requires only S. oneidensis to be viable, the biosensor can easily be deployed for long-term, autonomous monitoring, or for sensing higher concentrations of OP, merely by varying the ratio of the two bacteria, without the need for re-optimization. Overall, we have demonstrated and verified a unique electrochemical OP biosensing strategy based on engineered E. coli and S. oneidensis to degrade and detect highly toxic OPs in a single assay.

With the continued use of OP pesticides despite their toxicity, technologies to detect and degrade these chemicals are desperately needed. Our engineered cell mixture combined with electrochemical readout is the first example of a technology for specific recognition and destruction of this class of harmful compounds. Electrochemistry provides high sensitivity at a low cost, and engineered microbes offer unparalleled specificity. This work represents a paradigm shift in sensing and remediation through dual microbial engineering to control electron transfer in one organism with molecules generated from degradation by the other. Importantly, we anticipate this modular assembly to be readily applied to other classes of harmful contaminants.

### Materials and Methods

### Strain Engineering

OPH sequence. The organophosphate hydrolase (OPH) sequence used was parathion hydrolase from Pseudomonas diminuta (Uniprot ID: POA434). The sequence was codon-optimized for expression in E. coli and cloning-relevant restriction sites were removed. The OPH gene was synthesized by Twist Bioscience (South San Francisco, CA) as a fusion with the INPNC ice-nucleation sequence⁶³ on the N-terminus.

OPH-E. coli. The INPNC-OPH fusion was cloned into plasmid backbone pSKB3, previously described in [63]. pSKB3 is a variation of Novagen's pET-28a vector with the thrombin site exchanged for a TEV proteolysis site. The vector (pSKB3) and the insert (INPNC-OPH) were double digested with XhoI and NcoI for 35 minutes at 37° C. Digestion products were run on a TAE 1.6% agarose gel at 100 V for 30 minutes. Desired fragments were gel extracted using the Zymoclean™ Gel DNA Recovery Kit (Zymo Research). Purified DNA fragments were ligated using T4 DNA Ligase (NEB M0202). A 1:3 molar ratio of vector to insert and 50 ng of vector was used. Ligation mixture was transformed into chemically competent DH50α E. coli and plated on LB-agar kanamycin plates to select for positive transformants. A single colony was picked from the agar plate, inoculated in 5 mL LB-kanamycin, grown in a shaking incubator (250 rpm) for at least 16 hours at 37° C., and miniprepped using QIAprep™ Spin Miniprep Kit (Qiagen). The plasmid DNA was sequence-verified by sample submission to Genewiz (Cambridge, MA). Subsequently, the plasmid was transformed into chemically competent BL21 (DE3) E. coli to create the surface-expressed OPH-E. coli strain.

DmpR and PDmpR sequences. The sequence used for the p-NP-responsive transcription factor DmpR (dimethyl phenol regulator) was obtained from Pseudomonas spp. CF600 (Uniprot ID: Q06573). This sequence was modified with mutations Q10R and K117M because these mutations had been shown to improve the responsiveness of this transcription factor towards p-NP by seven-fold over the native protein.¹ The promoter sequence recognized by DmpR (P_{DmpR}) was obtained from the iGEM Registry of Standard Biological Parts. P_{DmpR} in our plasmid maps is identical to the Po promoter sequence including the ribosome binding site B0031 in part BBa_K1031221. DmpR and P_{DmpR} sequences were codon-optimized for expression in S. oneidensis and synthesized by Twist Bioscience (South San Francisco, CA).

### Preparation of Lyophilized E. coli

E. coli (engineered or wild-type) was grown overnight for 18-20 hours in 5 mL LB supplemented with 50 μg/mL kanamycin at 37° C. and 200 rpm from 25% frozen glycerol stocks (stored at −80° C.). The pre-culture was diluted to 0.1 OD₆₀₀ in 20 mL Terrific Broth (TB) supplemented with potassium phosphate buffer (17 mM KH₂PO_{4, 72} mM K₂HPO₄), 0.5% glucose and 50 μg/mL kanamycin. The culture was incubated at 37° C. and 200 rpm until OD₆₀₀ was approximately 0.8. The culture was induced using 100 μM IPTG and incubated at 18° C., 200 rpm for 20 hours. The cells were pelleted by centrifugation at 8942×g g for 3 minutes. The supernatant was discarded, and cells were washed twice by centrifugation and resuspension in a defined media. After the final wash, cells were diluted in the same defined media with 100 mM trehalose as a cryoprotectant to a final OD₆₀₀ of 0.1 or 2.0, depending upon the use. The defined media for resuspension was phosphate citrate buffer (pH 8.0) for colorimetric assays, phosphate buffered saline (pH 7.4) for fluorescence assays and MI minimal media (pH 7.0) for electrochemical measurements. 1 mL aliquots of the cell culture at specific OD₆₀₀ were flash frozen with liquid N2, lyophilized under vacuum and stored at −20° C. until further use. Before use, the lyophilized cells were reconstituted in sterile water.

### Bioelectrochemical Measurements

### Electrochemical Analysis

The electrochemical measurements were performed in a single-chamber three electrode bioreactor consisting of 1 cm² PW06 carbon cloth (Zoltek, St. Louis, MO) as a working electrode, Pt wire (Sigma Aldrich) as a counter electrode and AgCl/Ag (CH Instruments, Austin, TX, USA) as a reference electrode. 90 mL of M1 media modified from previous reports⁷⁷ was used for the electrode cultivation of S. oneidensis. The cells were provided with 18 mM sodium lactate as a carbon and electron source. The media was further supplemented with vitamins, minerals, and amino acids as previously-reported.⁷⁷ Riboflavin was, however, excluded from the vitamin solution to avoid riboflavin-induced electrochemical signals. The reactors were sealed with a rubber stopper with two needles to allow minimal air diffusion. Chronoamperometry measurements were made using a 16-channel potentiostat (Biologic, Seyssinet-pariset, France) or a 4-channel potentiostat (Admiral Instruments, Tempe, AZ). The working electrode potential was maintained at 0.4V vs AgCl/Ag reference electrode, thereby acting as an electron sink for the bacteria. A baseline current was first obtained at the applied potential prior to S. oneidensis inoculation. To study the effect of p-NP on the engineered S. oneidensis strain, the cells were first inoculated in the bioreactor to a final OD₆₀₀ of 0.8, followed by an injection of p-NP to a final concentration of 20 μM, at an interval of 1 hour. For co-culture experiments, after an hour of S. oneidensis inoculation, 1 mL of lyophilized E. coli cells in MI media were resuspended in sterile water and added to the bioreactor to a final OD₆₀₀ of 0.02. After another hour, the OP at the desired final concentration was injected. The current was measured for 24 hours after the p-nitrophenol or OP injection and normalized to the projected surface area of the carbon cloth electrode. The total charge produced by p-NP, or OP was determined by computing the area under the chronoamperometry curve from their time of introduction in the bioreactor up to 24 hours using OriginLab data analysis software (Northampton, MA).

### REFERENCES

(1) Thakur, M.; Medintz, I. L.; Walper, S. A. Enzymatic Bioremediation of Organophosphate Compounds—Progress and Remaining Challenges. Frontiers in Bioengineering and Biotechnology. 2019. p 289.
(2) DuBois, K. P.; Doull, J.; Salerno, P. R.; Coon, J. M. STUDIES ON THE TOXICITY AND MECHANISM OF ACTION OF P-NITROPHENYL DIETHYL THIONOPHOSPHATE (PARATHION). J. Pharmacol. Exp. Ther. 1949, 95 (1), 79 LP-91.
(3) Stokes, L.; Stark, A.; Marshall, E.; Narang, A. Neurotoxicity among Pesticide Applicators Exposed to Organophosphates. Occup. Environ. Med. 1995, 52 (10), 648-653. doi.org/10.1136/oem.52.10.648.
(4) Ruckart, P. Z.; Kakolewski, K.; Bove, F. J.; Kaye, W. E. Long-Term Neurobehavioral Health Effects of Methyl Parathion Exposure in Children in Mississippi and Ohio. Environ. Health Perspect. 2004, 112 (1), 46-51. doi.org/10.1289/ehp.6430.
(5) Costa, L. G. Organophosphorus Compounds at 80: Some Old and New Issues. Toxicol. Sci. 2018. 162 (1), 24-35. doi.org/10.1093/toxsci/kfx266.
(6) Franjesevic, A. J.; Sillart, S. B.; Beck, J. M.; Vyas, S.; Callam, C. S.; Hadad, C. M. Resurrection and Reactivation of Acetylcholinesterase and Butyrylcholinesterase. Chemistry 2019, 25 (21), 5337-5371. doi.org/10.1002/chem.201805075.
(7) Takamiya, K. Monitoring of Urinary Alkyl Phosphates in Pest Control Operators Exposed to Various Organophosphorus Insecticides. Bull. Environ. Contam. Toxicol. 1994, 52 (2), 190-195. doi.org/10.1007/BF00198487.
(8) Heudorf, U.; Angerer, J. Metabolites of Organophosphorous Insecticides in Urine Specimens from Inhabitants of a Residential Area. Environ. Res. 2001, 86 (1), 80-87. doi.org/doi.org/10.1006/enrs.2001.4237.
(9) Arcury, T. A.; Grzywacz, J. G.; Davis, S. W.; Barr, D. B.; Quandt, S. A. Organophosphorus Pesticide Urinary Metabolite Levels of Children in Farmworker Households in Eastern North Carolina. Am. J. Ind. Med. 2006, 49 (9), 751-760. doi.org/doi.org/10.1002/ajim.20354.
(10) Hertz-Picciotto, I.; Sass, J. B.; Engel, S.; Bennett, D. H.; Bradman, A.; Eskenazi, B.; Lanphear, B.; Whyatt, R. Organophosphate Exposures during Pregnancy and Child Neurodevelopment: Recommendations for Essential Policy Reforms. PLoS Med. 2018, 15 (10), 1-15. doi.org/10.1371/journal.pmed.1002671.
(11) Eddleston, M.; Phillips, M. R. Self Poisoning with Pesticides. BMJ 2004, 328 (7430), 42-44. doi.org/10.1136/bmj.328.7430.42.
(12) Eyer, P. The Role of Oximes in the Management of Organophosphorus Pesticide Poisoning. Toxicol. Rev. 2003, 22 (3), 165-190. doi.org/10.2165/00139709-200322030-00004.
(13) Karunarathne, A.; Gunnell, D.; Konradsen, F.; Eddleston, M. How Many Premature Deaths from Pesticide Suicide Have Occurred since the Agricultural Green Revolution? Clin. Toxicol. (Phila). 2020, 58 (4), 227-232. doi.org/10.1080/15563650.2019.1662433.
(14) Flounders, A. W.; Singh, A. K.; Volponi, J. V; Carichner, S. C.; Wally, K.; Simonian, A. S.; Wild, J. R.; Schoeniger, J. S. Development of Sensors for Direct Detection of Organophosphates.: Part II: Sol-Gel Modified Field Effect Transistor with Immobilized Organophosphate Hydrolase. Biosens. Bioelectron. 1999, 14 (8), 715-722. doi.org/doi.org/10.1016/S0956-5663(99)00045-7.
(15) Del Giudice, I.; Coppolecchia, R.; Merone, L.; Porzio, E.; Carusone, T. M.; Mandrich, L.; Worck, F.; Manco, G. An Efficient Thermostable Organophosphate Hydrolase and Its Application in Pesticide Decontamination. Biotechnol. Bioeng. 2016, 113 (4), 724-734. doi.org/doi.org/10.1002/bit.25843.
(16) Gopal, S.; Rastogi, V.; Ashman, W.; Mulbry, W. Mutagenesis of Organophosphorus Hydrolase to Enhance Hydrolysis of the Nerve Agent VX. Biochem. Biophys. Res. Commun. 2000, 279 (2), 516-519. doi.org/doi.org/10.1006/bbrc.2000.4004.
(17) Rastogi, V. K.; Defrank, J. J.; Cheng, T.; Wild, J. R. Enzymatic Hydrolysis of Russian-VX by Organophosphorus Hydrolase. Biochem. Biophys. Res. Commun. 1997, 241 (2), 294-296. doi.org/doi.org/10.1006/bbrc. 1997.7569.
(18) Kim, M.; Gkikas, M.; Huang, A.; Kang, J. W.; Suthiwangcharoen, N.; Nagarajan, R.; Olsen, B. D. Enhanced Activity and Stability of Organophosphorus Hydrolase via Interaction with an Amphiphilic Polymer. Chem. Commun. 2014, 50 (40), 5345-5348. doi.org/10.1039/C3CC47675H.
(19) LcJeune, K. E.; Wild, J. R.; Russell, A. J. Nerve Agents Degraded by Enzymatic Foams. Nature 1998, 395 (6697), 27-28. doi.org/10.1038/25634.
(20) Lu, H. D.; Wheeldon, I. R.; Banta, S. Catalytic Biomaterials: Engineering Organophosphate Hydrolase to Form Self-Assembling Enzymatic Hydrogels. Protein Eng. Des. Sel. 2010, 23 (7), 559-566. doi.org/10.1093/protein/gzq026.
(21) Lee, Y.; Stanish, I.; Rastogi, V.; Cheng, T.; Singh, A. Sustained Enzyme Activity of Organophosphorus Hydrolase in Polymer Encased Multilayer Assemblies.Langmuir 2003, 19 (4), 1330-1336. doi.org/10.1021/la0263965.
(22) Chen, B.; Lei, C.; Shin, Y.; Liu, J. Probing Mechanisms for Enzymatic Activity Enhancement of Organophosphorus Hydrolase in Functionalized Mesoporous Silica. Biochem. Biophys. Res. Commun. 2009. 390 (4), 1177-1181. doi.org/doi.org/10.1016/j.bbrc.2009.10.112.
(23) Lei, C.; Shin, Y.; Liu, J.; Ackerman, E. J. Entrapping Enzyme in a Functionalized Nanoporous Support. J. Am. Chem. Soc. 2002, 124 (38), 11242-11243. doi.org/10.1021/ja026855o.
(24) LeJeune, K. E.; Russell, A. J. Biocatalytic Nerve Agent Detoxification in Fire Fighting Foams. Biotechnol. Bioeng. 1999, 62 (6), 659-665. doi.org/doi.org/10.1002/(SICI)1097-0290(19990320)62:6<659::AID-BIT5>3.0.CO;2-N.
(25) Pedrosa, V. A.; Paliwal, S.; Balasubramanian, S.; Nepal, D.; Davis, V.; Wild, J.; Ramanculov, E.; Simonian, A. Enhanced Stability of Enzyme Organophosphate Hydrolase Interfaced on the Carbon Nanotubes. Colloids Surfaces B Biointerfaces 2010, 77 (1), 69-74. doi.org/doi.org/10.1016/j.colsurfb.2010.01.009.
(26) Mansce, A. H.; Chen, W.; Mulchandani, A. Detoxification of the Organophosphate Nerve Agent Coumaphos Using Organophosphorus Hydrolase Immobilized on Cellulose Materials. J. Ind. Microbiol. Biotechnol. 2005, 32 (11-12), 554-560. doi.org/10.1007/s10295-005-0059-y.
(27) Frančič, N.: Košak, A.; Lyagin. I.; Efremenko, E. N.; Lobnik, A. His6-OPH Enzyme-Based Bio-Hybrid Material for Organophosphate Detection. Anal. Bioanal. Chem. 2011, 401 (8), 2631-2638. doi.org/10.1007/s00216-011-5336-1.
(28) LeJeune, K. E.; Mesiano, A. J.; Bower, S. B.; Grimsley, J. K.; Wild, J. R.; Russell, A. J. Dramatically Stabilized Phosphotriesterase—Polymers for Nerve Agent Degradation. Biotechnol. Bioeng. 1997, 54 (2), 105-114. doi.org/doi.org/10.1002/(SICI)1097-0290(19970420)54:2<105::AID-BIT2>3.0.CO;2-P.
(29) Wei, W.; Du, J.; Li, J.; Yan, M.; Zhu, Q.; Jin, X.; Zhu, X.; Hu, Z.; Tang, Y.; Lu, Y. Construction of Robust Enzyme Nanocapsules for Effective Organophosphate Decontamination, Detoxification, and Protection. Adv. Mater. 2013. 25 (15), 2212-2218. doi.org/doi.org/10.1002/adma.201205138.
(30) Novikov, B. N.; Grimsley, J. K.; Kern, R. J.; Wild, J. R.; Wales, M. E. Improved Pharmacokinetics and Immunogenicity Profile of Organophosphorus Hydrolase by Chemical Modification with Polyethylene Glycol. J. Control. Release 2010, 146 (3), 318-325. doi.org/doi.org/10.1016/j.jconrel.2010.06.003.
(31) Sergeeva, V. S.; Efremenko, E. N.; Kazankov, G. M.; Gladilin, A. K.; Varfolomeev, S. D. Kinetic Behavior of Phosphotriesterase and Its Non-Covalent Complexes with Polyelectrolyte in Systems with Polar and Non-Polar Organic Solvents. Biotechnol. Tech. 1999. 13 (7), 479-483. doi.org/10.1023/A:1008985602921.
(32) Mulchandani. P.; Chen. W.; Mulchandani, A. Microbial Biosensor for Direct Determination of Nitrophenyl-Substituted Organophosphate Nerve Agents Using Genetically Engineered Moraxella Sp. Anal. Chim. Acta 2006. 568 (1-2), 217-221. doi.org/10.1016/j.aca.2005.11.063.
(33) Jha. R. K.; Kern. T. L.; Kim, Y.; Tesar, C.; Jedrzejczak, R.; Joachimiak, A.; Strauss, C. E. M. A Microbial Sensor for Organophosphate Hydrolysis Exploiting an Engineered Specificity Switch in a Transcription Factor. Nucleic Acids Res. 2016. 44 (17), 8490-8500. doi.org/10.1093/nar/gkw687.
(34) Gilbert. E. S.; Walker. A. W.; Kcasling. J. D. A Constructed Microbial Consortium for Biodegradation of the Organophosphorus Insecticide Parathion. Appl. Microbiol. Biotechnol. 2003. 61 (1). 77-81. doi.org/10.1007/s00253-002-1203-5.
(35) Jiang, J.; Zhang, R.; Li, R.; Gu. J.-D.; Li, S. Simultancous Biodegradation of Methyl Parathion and Carbofuran by a Genetically Engineered Microorganism Constructed by Mini-Tn5 Transposon. Biodegradation 2006. 18 (4), 403. doi.org/10.1007/s10532-006-9075-5.
(36) Liu, Z.; Hong. Q.; Xu, J.-H.; Jun. W.; Li, S.-P. Construction of a Genetically Engineered Microorganism for Degrading Organophosphate and Carbamate Pesticides. Int. Biodeterior. Biodegradation 2006. 58 (2), 65-69. doi.org/doi.org/10.1016/j.ibiod.2006.07.009.
(37) Hong, M. S.; Rainina, E.; Grimsley, J. K.; Dalc, B. E.; Wild. J. R. Neurotoxic Organophosphate Degradation with Polyvinyl Alcohol Gel-Immobilized Microbial Cells. Bioremediat. J. 1998. 2 (2), 145-157. doi.org/10.1080/10889869891214277.
(38) Wang, A. A.; Mulchandani, A.; Chen, W. Specific Adhesion to Cellulose and Hydrolysis of Organophosphate Nerve Agents by a Genetically Engineered &lt;Em&gt;Escherichia Coli&lt;/Em&gt; Strain with a Surface-Expressed Cellulose-Binding Domain and Organophosphorus Hydrolase. Appl. Environ. Microbiol. 2002. 68 (4), 1684 LP-1689. doi.org/10.1128/AEM.68.4.1684-1689.2002.
(39) Shimazu, M.; Mulchandani, A.; Chen, W. Cell Surface Display of Organophosphorus Hydrolase Using Ice Nucleation Protein. Biotechnol. Prog. 2001. 17 (1). 76-80. doi.org/doi.org/10.1021/bp0001563.
(40) Mulchandani, A.; Rajesh. Microbial Biosensors for Organophosphate Pesticides. Appl. Biochem. Biotechnol. 2011. 165 (2), 687-699. doi.org/10.1007/s12010-011-9288-x.
(41) Jaffrezic-Renault, N. New Trends in Biosensors for Organophosphorus Pesticides. Sensors. 2001. doi.org/10.3390/s10100060.
(42) Saida, F.; Uzan, M.; Odacrt, B.; Bontems, F. Expression of Highly Toxic Genes in E. Coli: Special Strategies and Genetic Tools. Current Protein and Peptide Science. pp 47-56.
(43) Ras, M. R.; Borrull, F.; Marcé, R. M. Sampling and Preconcentration Techniques for Determination of Volatile Organic Compounds in Air Samples. TrAC Trends Anal. Chem. 2009. 28 (3), 347-361. doi.org/doi.org/10.1016/j.trac.2008.10.009.
(44) Barker, Z.; Venkatchalam, V.; Martin, A. N.; Farquar, G. R.; Frank, M. Detecting Trace Pesticides in Real Time Using Single Particle Aerosol Mass Spectrometry. Anal. Chim. Acta 2010. 661 (2), 188-194. doi.org/10.1016/j.aca.2009.12.031.
(45) Peng, Y.; Fu, S.; Liu, H.; Lucia, L. A. Accurately Determining Esterase Activity via the Isosbestic Point of P-Nitrophenol. Bioresour. Vol 11, No 4 2016.
(46) Ngassa, G. B. P.; Tonlé, I. K.; Ngameni, E. Square Wave Voltammetric Detection by Direct Electroreduction of Paranitrophenol (PNP) Using an Organosmectite Film-Modified Glassy Carbon Electrode. Talanta 2016, 147, 547-555. doi.org/doi.org/10.1016/j.talanta.2015.10.030.
(47) Tapsoba, I.; Bourhis, S.; Feng, T.; Pontić, M. Sensitive and Selective Electrochemical Analysis of Methyl-Parathion (MPT) and 4-Nitrophenol (PNP) by a New Type p-NiTSPc/p-PPD Coated Carbon Fiber Microelectrode (CFME). Electroanalysis 2009, 21 (10), 1167-1176. doi.org/doi.org/10.1002/elan.200804529.
(48) Liu, Y.; Cao, X.; Liu, Z.; Sun, L.; Fang, G.; Liu, J.; Wang, S. Electrochemical Detection of Organophosphorus Pesticides Based on Amino Acids-Conjugated P3TAA-Modified Electrodes. Analyst 2020, 145 (24), 8068-8076. doi.org/10.1039/DOAN01838D.
(49) Qiu, L.; Lv, P.; Zhao, C.; Feng, X.; Fang, G.; Liu, J.; Wang, S. Electrochemical Detection of Organophosphorus Pesticides Based on Amino Acids Conjugated Nanoenzyme Modified Electrodes. Sensors Actuators B Chem. 2019, 286, 386-393. doi.org/doi.org/10.1016/j.snb.2019.02.007.
(50) Adkins, J. A.; Bochle, K.; Friend, C.; Chamberlain, B.; Bisha, B.; Henry, C. S. Colorimetric and Electrochemical Bacteria Detection Using Printed Paper- and Transparency-Based Analytic Devices. Anal. Chem. 2017. 89 (6), 3613-3621. doi.org/10.1021/acs.analchem.6b05009.
(51) RU, S.-P.; WU, J.; YING, Y.-B.; JI, F. Electrochemical Detection of Alkaline Phosphatase Using Ionic Liquid Modified Carbon Nanotubes Electrode. Chinese J. Anal. Chem. 2012, 40 (6), 835-840. doi.org/doi.org/10.1016/S1872-2040(11)60552-5.
(52) Kim, I.; Kim, G. H.; Kim, C. S.; Cha, H. J.; Lim, G. Optical Detection of Paraoxon Using Single-Walled Carbon Nanotube Films with Attached Organophosphorus Hydrolase-Expressed Escherichia Coli. Sensors (Basel). 2015, 15 (6), 12513-12525. doi.org/10.3390/s150612513.
(53) Hayat, A.; Marty, J. L. Disposable Screen Printed Electrochemical Sensors: Tools for Environmental Monitoring. Sensors (Basel). 2014, 14 (6), 10432-10453. doi.org/10.3390/s140610432.
(54) D′Souza, S. F. Microbial Biosensors. Biosens. Bioelectron. 2001, 16 (6), 337-353. doi.org/doi.org/10.1016/S0956-5663(01)00125-7.
(55) Belkin, S. Microbial Whole-Cell Sensing Systems of Environmental Pollutants. Curr. Opin. Microbiol. 2003, 6 (3), 206-212. doi.org/doi.org/10.1016/S1369-5274(03)00059-6.
(56) Joshi, N.; Wang, X.; Montgomery, L.; Elfick, A.; French, C. E. Novel Approaches to Biosensors for Detection of Arsenic in Drinking Water. Desalination 2009, 248 (1), 517-523. doi.org/doi.org/10.1016/j.desal.2008.05.096.
(57) Wang, B.; Barahona, M.; Buck, M. A Modular Cell-Based Biosensor Using Engineered Genetic Logic Circuits to Detect and Integrate Multiple Environmental Signals. Biosens. Bioelectron. 2013, 40 (1), 368-376. doi.org/doi.org/10.1016/j.bios.2012.08.011.
(58) Sagi, E.; Hever, N.; Rosen, R.; Bartolome, A. J.; Rajan Premkumar, J.; Ulber, R.; Lev, O.; Scheper, T.; Belkin, S. Fluorescence and Bioluminescence Reporter Functions in Genetically Modified Bacterial Sensor Strains. Sensors Actuators B Chem. 2003, 90 (1), 2-8. doi.org/doi.org/10.1016/S0925-4005(03)00014-5.
(59) Ramanathan, S.; Shi, W.; Rosen, B. P.; Daunert, S. Sensing Antimonite and Arsenite at the Subattomole Level with Genetically Engineered Bioluminescent Bacteria. Anal. Chem. 1997, 69 (16), 3380-3384. doi.org/10.1021/ac970111p.
(60) Breuer, M.; Rosso, K. M.; Blumberger, J.; Butt, J. N. Multi-Haem Cytochromes in Shewanella Oneidensis MR-1: Structures, Functions and Opportunities. J. R. Soc. Interface 2015, 12 (102). doi.org/10.1098/rsif.2014.1117.
(61) El-Naggar, M. Y.; Wanger, G.; Leung, K. M.; Yuzvinsky, T. D.; Southam, G.; Yang, J.; Lau, W. M.; Nealson, K. H.; Gorby, Y. A. Electrical Transport along Bacterial Nanowires from Shewanella Oneidensis MR-1. Proc. Natl. Acad. Sci. U. S. A. 2010, 107 (42), 18127-18131. doi.org/10.1073/pnas. 1004880107.
(62) Von Canstein, H.; Ogawa, J.; Shimizu, S.; Lloyd, J. R. Secretion of Flavins by Shewanella Species and Their Role in Extracellular Electron Transfer. Appl. Environ. Microbiol. 2008, 74 (3), 615-623. doi.org/10.1128/AEM.01387-07.
(63) Marsili, E.; Baron, D. B.; Shikhare, I. D.; Coursolle, D.; Gralnick, J. A.; Bond, D. R. Shewanella Secretes Flavins That Mediate Extracellular Electron Transfer. Proc. Natl. Acad. Sci. U. S. A. 2008, 105 (10), 3968-3973. doi.org/10.1073/pnas.0710525105.
(64) Furst, A. L.; Hoepker, A. C.; Francis, M. B. Quantifying Hormone Disruptors with an Engineered Bacterial Biosensor. ACS Cent. Sci. 2017, 3 (2), 110-116. doi.org/10.1021/acscentsci.6b00322.
(65) Kaur, J.; Singh, P. K. Enzyme-Based Optical Biosensors for Organophosphate Class of Pesticide Detection. Phys. Chem. Chem. Phys. 2020, 22 (27), 15105-15119. doi.org/10.1039/d0cp01647k.
(66) Dumas, D. P.; Caldwell, S. R.; Wild, J. R.; Raushel, F. M. Purification and Properties of the Phosphotriesterase from Pseudomonas Diminuta. J. Biol. Chem. 1989. 264 (33), 19659-19665. doi.org/10.1016/s0021-9258(19)47164-0.
(67) DAVIS, R. J. Viability and Behavior of Lyophilized Cultures after Storage for Twenty-One Years. J. Bacteriol. 1963, 85, 486-487. doi.org/10.1128/jb.85.2.486-487.1963.
(68) Bowers, G. N. J.; McComb, R. B.; Christensen, R. G.; Schaffer, R. High-Purity 4-Nitrophenol: Purification, Characterization, and Specifications for Use as a Spectrophotometric Reference Material. Clin. Chem. 1980, 26 (6), 724-729.
(69) Richins, R. D.; Mulchandani, A.; Chen, W. Enzymatic Characterization of Fusion Enzymes. Biotechnology 2000.
(70) Efremenko, E. N.; Sergeeva, V. S. Organophosphate Hydrolase-An Enzyme Catalyzing Degradation of Phosphorus-Containing Toxins and Pesticides. Russ. Chem. Bull. 2001, 50 (10), 1826-1832. doi.org/10.1023/A:1014377912147.
(71) Wise, A. A.; Kuske, C. R. Generation of Novel Bacterial Regulatory Proteins That Detect Priority Pollutant Phenols. Appl. Environ. Microbiol. 2000, 66 (1), 163-169. doi.org/10.1128/AEM.66.1.163-169.2000.
(72) Webster, D. P.; TerAvest, M. A.; Doud, D. F. R.; Chakravorty, A.; Holmes, E. C.; Radens, C. M.; Sureka, S.; Gralnick, J. A.; Angenent, L. T. An Arsenic-Specific Biosensor with Genetically Engineered Shewanella Oneidensis in a Bioelectrochemical System. Biosens. Bioelectron. 2014, 62, 320-324. doi.org/doi.org/10.1016/j.bios.2014.07.003.
(73) Teravest, M. A.; Rosenbaum, M. A.; Kotloski, N. J.; Gralnick, J. A.; Angenent, L. T. Oxygen Allows Shewanella Oneidensis MR-1 to Overcome Mediator Washout in a Continuously Fed Bioelectrochemical System. Biotechnol. Bioeng. 2014, 111 (4), 692-699. doi.org/10.1002/bit.25128.
(74) Kafi, A. K. M.; Chen, A. A Novel Amperometric Biosensor for the Detection of Nitrophenol. Talanta 2009, 79 (1), 97-102. doi.org/10.1016/j.talanta.2009.03.015.
(75) Santhiago, M.; Henry, C. S.; Kubota, L. T. Low Cost, Simple Three Dimensional Electrochemical Paper-Based Analytical Device for Determination of p-Nitrophenol. Electrochim. Acta 2014, 130, 771-777. doi.org/10.1016/j.electacta.2014.03.109.
(76) Das, J.; Aziz, M. A.; Yang, H. A Nanocatalyst-Based Assay for Proteins: DNA-Free Ultrasensitive Electrochemical Detection Using Catalytic Reduction of p-Nitrophenol by Gold-Nanoparticle Labels. J. Am. Chem. Soc. 2006, 128 (50), 16022-16023. doi.org/10.1021/ja0672167.
(77) Bretschger, O.; Obraztsova, A.; Sturm, C. A.; In, S. C.; Gorby, Y. A.; Reed, S. B.; Culley, D. E.; Rcardon, C. L.; Barua, S.; Romine, M. F.; et al. Current Production and Metal Oxide Reduction by Shewanella Oncidensis MR-1 Wild Type and Mutants. Appl. Environ. Microbiol. 2007, 73 (21), 7003-7012. doi.org/10.1128/AEM.01087-07.

### Supplemental Information

### 1. Strain Engineering

p-NP-responsive strains: The DmpR gene sequence and the P_{DmpR} sequence were cloned into the same vector. DmpR was placed under the control of the LacI promoter to ensure constitutive expression while the P_{DmpR} sequence was placed in front of the gene of interest (either sfGFP or CymA). The cloning was performed using a four-part Gibson assembly strategy.¹ The vector pieces were amplified by PCR from plasmids pCD7sfGFP and pCD26r4 (these plasmids were gifts from the Keitz Lab). The pCD plasmids have a ColE1 origin of replication, kanamycin resistance, a lacI repressor constitutively expressed, and either sfGFP or CymA under the control of an IPTG-inducible promoter. We designed primers such that DmpR would replace the lacI repressor and P_{DmpR} would replace the IPTG-inducible promoter. Thus, each Gibson Assembly reaction had four fragments: 1) the vector containing the gene of interest, origin of replication, antibiotic resistance, etc., 2) P_{DmpR}, 3) P_{lacI}, and 4) DmpR. PCR with Phusion™ High-Fidelity DNA Polymerase (NEB: M0530) in HF buffer was used to generate DNA fragments. The PCR parameters were: initial denaturation at 98° C. for 30 s followed by thirty-five cycles of 1) 98° C. for 10 s, 2) 61° C. for 30 s, and 3) 72° C. for 2 mins 30 s, followed by a final extension at 72° C. for 10 minutes. PCR products were run on a 1.6% agarose gel at 100 V for 30 minutes. Desired fragments were gel extracted using the Zymoclean™ Gel DNA Recovery Kit (Zymo Research). Gibson Assembly reactions were performed in a 20 μL reaction volume using a 1:3 molar ratio of vector to insert. Gibson Assembly master mix was prepared in house and contained ISO buffer, T5 exonuclease, Phusion™ polymerase, and Taq DNA ligase. Reactions were performed at 50° C. for 1 hour. After incubation, 10 μL of Gibson assembly reaction was transformed into chemically competent DH5α E. coli and plated on LB-agar kanamycin plates to select for positive transformants. Sequence verification of correctly cloned plasmids was performed as described above. After sequence verification, plasmids were transformed by electroporation into the appropriate strains of S. oneidensis. Electroporation protocol was adapted from Dundas et al.² In short, S. oneidensis cells were made competent by washing 3× and resuspending in 10% glycerol solution. After addition of ˜100 ng DNA, electroporation was performed in a BTX Harvard Apparatus ECM 399 Electroporation System using 1 mm electroporation cuvettes at 1250 V. Cells are recovered for 2 hours by shaking at 30° C. at 200 rpm and plated on LB-kanamycin (25 μg/ml) plates for selection. The plasmid containing P_{DmpR}>sfGFP was transformed into wildtype S. oneidensis MR-1 (gift from the Keitz Lab), and the plasmid containing P_{DmpR}>cymA was transformed into the genetic knockout strain S. oneidensis MR-1 ΔcymA (gift from the Keitz Lab).

### 2. Colorimetric Assay for Organophosphate Degradation Using Lyophilized OPH-E. coli

The colorimetric assay was performed in a 96-well plate using a multi-mode microplate reader (BioTek, Winooski, VT). Lyophilized OPH-E. coli cells in phosphate citrate (PC, 50 mM Na₂HPO₄, 9.5 μM citric acid monohydrate, pH=8.0) buffer were reconstituted in sterile water and diluted to a final OD₆₀₀ of 0.02 in 200 μL PC buffer. Just before the measurement, organophosphates (OP), including paraoxon and parathion were added from 100% MeOH stock solutions to the experimental wells at different concentrations keeping the final MeOH concentration at 0.1% (v/v). For paraoxon-methyl, the stock solutions were made of 10% MeOH in water, making the final MeOH concentration in the experimental wells was 0.01% (v/v). Controls with only OPH-E. coli cells and only the corresponding OP were also included. P-NP production was monitored by measuring absorbance at 400 nm over 2 hours at 27° C. Standard curves generated from known concentrations of p-NP were used to convert the absorbance values to p-NP concentrations. To determine the effect of enzyme concentration on OP degradation, absorbance at 400 nm was measured at a constant paraoxon concentration (25 μM) and varying cell concentrations starting from final OD₆₀₀ 0.04 and serially diluting six times by half. Parameters of enzyme kinetics were determined by fitting the data to the Michaelis-Menten equation using OriginLab data analysis software (Northampton, MA).

### 3. GFP Fluorescence Assay for Monoculture and Co-culture Experiments

Engineered S. oneidensis strain was grown overnight for 18-20 hours in 50 ml LB supplemented with 25 μg/mL kanamycin at 30° C. and 200 rpm from 25% frozen glycerol stocks (stored at −80° C.). The pre-culture was diluted in 30 mL Terrific Broth (TB) supplemented with potassium phosphate buffer (17 mM KH₂PO₄, 72 mM K₂HPO₄), and 25 μg/mL kanamycin to 0.1 OD₆₀₀ and incubated at 30° C. at 200 rpm until the OD₆₀₀ reached 0.8.

To determine the effect of p-NP on bacteria, the culture was split into 10 tubes and induced with increasing concentrations of p-NP ranging from 0 to 200 μM. The incubation was continued at 30° C. and 200 rpm for 24 hours. Cells were pelleted from 100 μL of the cultures by centrifugation at 14,265×g for 2 minutes. The supernatant was discarded, and the cells were washed twice by centrifugation and resuspension with PBS (pH 7.4). The cells were then pipetted in a 96-well plate at 1/10^{th} dilution in PBS and the fluorescence was measured in a microplate reader (BioTek, Winooski, VT) at excitation λ485 nm and emission λ510 nm.

For co-culture measurements, lyophilized OPH-E. coli cells in PBS were reconstituted in sterile water and inoculated to OD₆₀₀ 0.02 with engineered S. oneidensis culture in TB at OD₆₀₀ 0.8. OPs at different final concentrations were added to the co-cultures and incubated at 30° C., 200 rpm for 48 hours. Periodically, 100 μl of cell aliquots were withdrawn from the cultures; cells were pelleted and washed twice by centrifugation and resuspension in PBS at 14,265×g for 2 minutes, and the fluorescence was measured as described above. In both monoculture and co-culture assays, the fluorescence was normalized with OD600.

### 4. Bioelectrochemical Measurements

S. oneidensis (engineered or wild-type) was grown overnight for 18-20 hours in 50 ml LB supplemented with 25 mg/mL kanamycin at 30° C. and 200 rpm from 25% frozen glycerol stocks (stored at −80° C.). The cells were pelleted by centrifugation at 11,940×g for 5 minutes. The supernatant was discarded, and the cells were washed twice by centrifugation and resuspension in M1 minimal buffer adapted from previous reports³ containing 50 mM sodium salt of PIPES buffer, 28 mM ammonium chloride, 1.34 mM potassium chloride, and 4.35 mM sodium phosphate monobasic. After the final wash, the cells were resuspended in 1 ml M1 media and inoculated in bioreactors for electrochemical measurements.

### REFERENCES

ADDIN Mendeley Bibliography CSL_BIBLIOGRAPHY 1 D. G. Gibson, L. Young, R. Y. Chuang, J. C. Venter, C. A. Hutchison and H. O. Smith, Nat. Methods, 2009, 6, 343-345.
2 C. M. Dundas, D. J. F. Walker and B. K. Keitz, ACS Synth. Biol., 2020, 9, 2301-2315.
3 O. Bretschger, A. Obraztsova, C. A. Sturm, S. C. In, Y. A. Gorby, S. B. Reed, D. E. Culley, C. L. Reardon, S. Barua, M. F. Romine, J. Zhou, A. S. Beliaev, R. Bouhenni, D. Saffarini, F. Mansfeld, B. H. Kim, J. K. Fredrickson and K. H. Nealson, Appl. Environ. Microbiol., 2007, 73, 7003-7012.

1. A recombinant microbial cell displaying on its surface a non-native protein capable of degrading an organophosphate, wherein the recombinant microbial cell has inhibited replication.

2. The recombinant microbial cell of claim 1, wherein the recombinant cell is treated to reduce its ability to replicate by chemical treatment including but not limited to treatment with sodium azide or by lyophilization.

3.-5. (canceled)

6. The recombinant microbial cell of claim 1, wherein the protein comprises a phosphate hydrolase, beta-lactamases-methyl parathion hydrolase, lactonase, or a phosphate triesterase, or an enzymatically active fragment thereof.

7. The recombinant microbial cell of claim 1, wherein the protein is selected from the group consisting of an organophosphate hydrolase (OPH), OP (organophosphate)-degrading enzyme from Agrobacterium radiobacter (OpdA), phosphotriesterase (PTE), methyl parathion hydrolase (MPH), and SsoPOX, or an enzymatically active fragment thereof.

8. The recombinant microbial cell of claim 1, wherein the protein comprises an amino acid sequence at least 75%, 80%, 85%, 90%, 91%, 92%, 93%, 94%, 95%, 96%, 97%, 98%, 99%, or 100% identical to the amino acid sequence of SEQ ID NO: 1 or 3-5, wherein residues in parentheses are optional and may be present or absent.

9. The recombinant microbial cell of claim 1, wherein the protein further comprise one or more domain that facilitates microbial cell surface expression of the protein.

10. (canceled)

11. The recombinant microbial cell of claim 9, wherein the domain comprises an amino acid sequence at least 75%, 80%, 85%, 90%, 91%, 92%, 93%, 94%, 95%, 96%, 97%, 98%, 99%, or 100% identical to the amino acid sequence of SEQ ID NO:6 or 7, wherein residues in parentheses are optional and may be present or absent, and when present may be substituted with any other amino acid linker of the same or different length and amino acid composition

12. The recombinant microbial cell of claim 1, wherein the protein comprises an amino acid sequence at least 75%, 80%, 85%, 90%, 91%, 92%, 93%, 94%, 95%, 96%, 97%, 98%, 99%, or 100% identical to the amino acid sequence of SEQ ID NO:2, wherein the amino acid residues in parentheses are optional and may be present or absent, and when present may be substituted with any other amino acid linker of the same or different length and amino acid composition.

13. The recombinant microbial cell of claim 1, wherein the protein is encoded by a nucleic acid coding sequence present on a plasmid in the microbial cell, wherein the nucleic acid is operatively linked to control sequence capable of promoting expression of the nucleic acid.

14. The recombinant microbial cell of claim 1, wherein the microbial cell is a bacteria.

15. The recombinant microbial cell of claim 1, wherein the bacteria is E.coli.

16. The recombinant microbial cell of claim 1, wherein the cell is E. coli displaying an enzymatically active portion of parathion hydrolase from Pseudomonas diminuta on its surface.

17. The recombinant microbial cell of claim 16, wherein the cell displays an enzymatically active portion of the amino acid sequence of SEQ ID NO:1 or SEQ ID NO:2 on its surface.

18.-19. (canceled)

20. A recombinant microbial cell engineered to be capable of expressing a non-native transcription factor that activates a non-native promoter in response to an organophosphate degradation product; wherein the non-native promoter is operatively linked to a nucleic acid encoding a reporter protein, wherein activity of the reporter protein can be detected.

21. The microbial cell of claim 20, wherein the non-native transcription factor comprises DmpR, or variants thereof, or wherein the non-native transcription factor comprises any small molecule-responsive transcription factor.

22. The microbial cell of claim 21, wherein the transcription factor comprises an amino acid sequence at least 75%, 80%, 85%, 90%, 91%, 92%, 93%, 94%, 95%, 96%, 97%, 98%, 99%, or 100% identical to the amino acid sequence of SEQ ID NO: 8, optionally modified with mutations Q10R and K117M, wherein residues in parentheses are optional and may be present or absent.

23. The recombinant microbial cell of claim 20, comprising a plasmid that comprises (a) a nucleic acid encoding the non-native transcription factor and (b) the non-native promoter operatively linked to the reporter protein.

24.-31. (canceled)

32. A kit comprising:
(a) a first recombinant microbial cell comprising the recombinant microbial cell of claim 1; and
(b) a second recombinant microbial cell engineered to be capable of expressing a non-native transcription factor that activates a non-native promoter in response to an organophosphate degradation product; wherein the non-native promoter is operatively linked to a nucleic acid encoding a reporter protein, wherein activity of the reporter protein can be detected.

33. (canceled)

34. A method for degrading organophosphates, comprising contacting a source suspected of containing organophosphates with a first recombinant microbial cell of claim 1, thereby degrading organophosphates present in the source.

35. A method for degradation of organophosphates and detection of organophosphate degradation products, comprising
(a) contacting a source suspected of containing organophosphates with (i) a first recombinant microbial cell of claim 1, thereby degrading organophosphates present in the source to generate organophosphate degradation products; and
(ii) a second recombinant microbial cell engineered to be capable of expressing a non-native transcription factor that activates a non-native promoter in response to an organophosphate degradation product; wherein the non-native promoter is operatively linked to a nucleic acid encoding a reporter protein, wherein activity of the reporter protein can be detected;
(b) detecting reporter protein signal, wherein the reporter protein signal provides a measure of organophosphate degradation products in the source.

36.-38. (canceled)
